(19) United States
(12) Patent Application Publication
(10) Pub. No.: US 2026/0276575 A1
(43) Pub. Date: Sep. 17, 2026

(54) PEPTIDE BASED BRIDGES FOR MOLECULAR SENSORS AND METHODS FOR USE THEREOF

(71) Applicant: Roswell ME, Inc., San Diego, CA (US)

(72) Inventors: Sinha Kumar Prem, San Diego, CA (US); Krista M. Cosert, San Diego, CA (US); Jose D. Rizo, San Diego, CA (US); Teresa Jones, San Diego, CA (US); Carl W. Fuller, San Diego, CA (US); Barry Merriman, San Diego, CA (US); Paul Mola, San Diego, CA (US)

(21) Appl. No.: 18/843,280

(22) PCT Filed: Mar. 1, 2023
(86) PCT No.: PCT/US2023/014291

§ 371 Date: Sep. 2, 2024

Related U.S. Application Data
(63) Continuation of application No. PCT/US2020/013218, filed on Jan. 10, 2020.
(60) Provisional application No. 63/315,517, filed on Mar. 1, 2022.
(60) Provisional application No. 63/344,746, filed on May 23, 2022.

(51) Int. Cl. G01N 27/327; C12Q 1/00; C12Q 1/48; C12Q 1/6869

(52) U.S. Cl. CPC G01N 27/3271; C12Q 1/001; C12Q 1/48; C12Q 1/6869; G01N 27/3275

(57) ABSTRACT

In various embodiments alpha helical peptide-based bridges for molecular biosensing “on-chip” are disclosed. The Peptide-based bridges serve as common bridges for great diversity of biosensing applications and targets including nucleic acids, proteins, antigens, antibodies, small molecules. The primary sensor element is preferably a “molecular wire” typically comprised of 10-45 nm long alpha-helical peptide integrated into a current monitoring circuit. The engineered peptide may contain a central conjugation site for attachment of various probe molecules including nucleic acids, proteins, antigens, antibodies. The probe-containing bridge empowers the sensor to detect interactions with specific target molecules.

### REFERENCE TO CROSS-RELATED APPLICATIONS

This application claims priority to, and the benefit of, U.S. Provisional Patent Application Ser. No. 63/315,517 by Prem Sinha et al., entitled ‘Peptide-Based Bridges’ filed on Mar. 1, 2022 and U.S. Provisional Patent Application Ser. No. 63/344,746 by Prem Sinha et al., entitled ‘Peptide-Based Bridges’ filed on May 23, 2022, and WO/2020/146823A9 by Barry Merriman et al., entitled ‘Conductive Synthetic Peptides for Molecular Electronics’, filed on Jan. 10, 2020, the disclosures of which are incorporated herein by reference in their entireties for all purposes.

### SEQUENCE LISTING

The instant application contains a Sequence Listing which has been submitted electronically in XML format and is hereby incorporated by reference in its entirety. Said XML copy, created on Apr. 21, 2026, is named ROS-2100-PCT-US_SL.xml and is 67,342 bytes in size.

### FIELD

The present disclosure is generally directed to molecular sensors in which a probe is bound to an amino acid bridge molecule in a molecular circuit and binding of a target or ligand to the probe is detectible by monitoring at least one parameter of the molecular circuit.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

The broad field of molecular electronics was introduced in the 1970's by Aviram and Ratner. Molecular electronics achieves the ultimate scaling down of electrical circuits by using single molecules as circuit components. Molecular circuits comprising single molecule components can function diversely as switches, rectifiers, actuators and sensors, depending on the nature of the molecule. Of particular interest is the application of such circuits as sensors, where molecular interactions provide a basis for single molecule sensing. In particular, informative current changes could include an increase, or decrease, a pulse, or other time variation in the current.

Notwithstanding the achievements in the field of molecular electronics, new molecular circuits that can function as molecular sensors are still needed. The need exists for simple and easy to manufacture molecular sensors. The need still also exists for improved single molecule systems that can yield molecular information with greater signal-to-noise ratios such that signals truly indicative of molecular interactions are distinguishable from non-informative noise. The inventions described herein meet these unsolved challenges and needs.

As described in detail herein below, novel embodiments of the invention described herein.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

The present disclosure generally relates to sensors, systems including the sensors, and to methods of using the sensors and systems. In various embodiments, binding probe-based circuits are disclosed. Exemplary sensors can be used to, for example, detect the binding of a molecule of interest (herein encompassed by the term ‘target) with the probe or a binding partner or ligand of the probe of the sensor. While the ways in which various embodiments of the disclosure address the drawbacks of the prior art sensors are discussed in more detail below, in general, the disclosure provides sensors that are relatively easy and inexpensive to manufacture.

This invention relates to alpha helical peptide-based bridges for molecular biosensing “on-chip”. The Peptide-based bridges serve as common bridges for great diversity of biosensing applications and targets including nucleic acids, proteins, antigens, antibodies, small molecules. The primary sensor element is preferably a “molecular wire”, also referred to as a bridge or current carrying molecular structure herein, is in selected embodiments comprised of 10-45 nm long alpha-helical peptide integrated into a current monitoring circuit. The engineered peptide may contain a central conjugation site for attachment of various probe molecules including nucleic acids, proteins, antigens, or antibodies. The probe-containing bridge empowers the sensor to detect interactions with specific target molecules. The current through the molecular bridge is typically monitored under an applied DC voltage. The detected signals are picoampere current levels with millisecond temporal resolution generated by transient probe-target molecular interaction.

Exemplary sensors comprise i) a molecular wire or current carrying molecular structure comprising a metal on its surface that is coupled, conjugated, or otherwise attached to ii) a bridge molecule, where the bridge molecule is coupled, conjugated, or otherwise attached to iii) a probe molecule, wherein a circuit is formed that is capable of detecting and obtaining detailed information about the binding of the probe to ligand or binding partner. Exemplary sensors may further include one or more linker, such as, for example, a chemical linker, a biopolymer (e.g. a peptide or a modified peptide chain), or a combination thereof that facilitate the coupling of the probe to the bridge molecule.

In various embodiments, the bridge molecule is a protein or peptide, such as a peptide alpha-helix. The peptide-based bridges disclosed herein are designed with several important criteria, which include the length of the peptide, the helicity of the peptide, the conductivity of the peptide, the introduction of one or more anchors or metal binding motifs, and the compatibility of the peptide with the specific metal surface used in the sensor. In the selection of peptides, the stability, purity, and yield are also factors considered in the selection. The peptides are evaluated herein by measuring conductivity and CD spectroscopy to determine helicity.

It is preferred that the peptides can be readily bio-conjugated to the probe molecule. A peptide alpha-helix bridge molecule may comprise one or more non-natural amino acid, including at the N and C termini. In some embodiments, the amino acid sequences of a peptide-based bridge molecule have a Cysteine (Cys, C), Lysine (Lys, K), or Tyrosine (Tyr, T) residue that is used for bioconjugation to a probe or linker that is also coupled to a probe. The types of alpha-helices of the peptide-based bridge molecule may include sequences EA3R (SEQ ID NO: 12), PTSTGQA (SEQ ID NO:10), YAREY (SEQ ID NO: 16), Pilin-inspired sequences, salt-bridge helices, and conductive peptides. The length of peptides used in the invention in some preferred embodiments is in the range of about 175 to about 275 amino acids in length, more generally peptides used herein are in the range of about 100 to about 400 amino acids in length; however longer and shorter peptides are envisioned. In some embodiments, alpha helix peptide bridges have anchors at one or both ends of the peptides, where said anchors may comprise metal binding motifs, thiol-based motifs, or specific amino acids at both termini for conjugation with modified metal surfaces.

Exemplary peptide alpha-helix bridges include the following, as well the peptides below having up to one, two, three, four, or five conservative amino acid substitutions or deletions that have been tested to retain functionality:

(SEQ ID NO: 18)
MDYKDDDDKGSGSGSQQSWPISGSGQQSWPISGSGQQSWPISGSGAE
AAAREAAAREAAAREAAAREAAAREAAAREAAAREAAAREAAAREAA
AREAAAREAAAREAAAREAAAREAAAREAAAREACAREAAAREAAAR
EAAAREAAAREAAAREAAAREAAAREAAAREAAAREAAAREAAAREA
AAREAAAREAAAREAAAREAAARAGSGQQSWPISGSGQQSWPISGSG
QQSWPIS
(SEQ ID NO: 19)
MGSGQQSWPISGSGQQSWPISGSGQQSWPISGSGAEAAAREAAAREA
AAREAAAREAAAREAAAREAAAREAAAREAAAREAAAREAAAREAAA
REAAAREAAAREAAAREAAAREAAAREAAAREAAAREAAAREAAARE
AAAREAAAREAAAREAAAREAAAREACAREAAAREAAAREAAAREAA
AREAAAREAAAREAAAREAAAREAAAREAAAREAAAREAAAREAAAR
EAAAREAAAREAAAREAAAREAAAREAAAREAAAREAAAREAAAREA
AAREAAAREAAAREAAARAGSGQQSWPISGSGQQSWPISGSGQQSWP
ISGSGENLYFQGMGSGHHHHHH
(SEQ ID NO: 20)
MDYKDDDDKGSGENLYFQGMGSGKHFYWMGSGQQSWPISGSGHFYWM
GSGTLHVSSYGSGFYWMGSGYEAYAREAYAREAYAREAAAREAAARE
AAAREAYAREAYAREAYAREAAAREAAAREAAAREAYAREAYAREAY
AREAAAREAAAREACAREAAAREAAAREAYAREAYAREAYAREAAAR
EAAAREAAAREAYAREAYAREAYAREAAAREAAAREAAAREAYAREA
YAREAYARYGSGMHFYWGSGTLHVSSYGSGMHFYWGSGQQSWPISGS
GKMHFYWGSGHHHHHH

In certain embodiments, the bridge molecule comprises one or more repeats of a sub-sequence of the Pilin protein from Geobacter sulfurreducens. Exemplary peptide alpha-helix bridges include the following, as well the peptides below having up to one, two, three, four, or five conservative amino acid substitutions or deletions that have been tested to retain functionality:

(SEQ ID NO: 21)
MDYKDDDDKGSGENLYFQGMGSGKHFYWMGSGQQSWPISGSGHFY
WMGSGTLHVSSYGSGFYWMGSGAIPQFSAYRVKAYNSAASSDLRN
LKTALAIPQFSAYRVKAYNSAASSDLRNLKTALAIPQFSAYRVKA
YNSAASSDLRNLKTALAIPQFSAYRVKAYNSAASSDLRNLKTCLA
IPQFSAYRVKAYNSAASSDLRNLKTALAIPQFSAYRVKAYNSAAS
SDLRNLKTALAIPQFSAYRVKAYNSAASSDLRNLKTALAIPQFSA
YRVKAYNSAASSDLRNLKTALGSGMHFYWGSGTLHVSSYGSGMHF
YWGSGQQSWPISGSGKMHFYWGSGHHHHHH
(SEQ ID NO: 22)
ENLYFQGMGSGMGSGCTLHVSSYCGSGCTLHVSSYCGSGCTLHVS
SYCGSGAAIPQFSAYRVRAYNSEAAAREAAAREAAARAIPQFSAY
RVRAYNSEAAAREAAAREAAARAIPQFSYRVRAYNSEAAAREAAA
REAKAREAAAREAAARAIPQFSAYRVRAYNSEAAAREAAAREAAA
RAIPQFSAYRVRAYNSEAAAREAAAREAAARAIPQFSAYRVRAYN
SAGSGCTLHVSSYCGSGCTLHVSSYCGSGCTLHVSSYCLEVLFQG
P GSGHHHHHH

Exemplary sensors include a probe attached to the bridge molecule. In various embodiments, the probe of the sensor comprises an enzyme, such as, for example, a DNA polymerase. In some embodiments, the probe is an antibody or binding portion thereof, or alternatively the probe of the sensor comprises one member of a ligand and binding partner pair. In some cases, the probe may comprise a DNA polymerase, such as, for example, Phi29, Bst, PolI, or a mutant thereof. The probe may also be a nucleic acid that can bind its complement or various proteins.

In some embodiments of the present disclosure, a sensor includes a source electrode; a drain electrode spaced apart from the source electrode by a sensor gap; wherein the source and drain electrode cooperate to form an electrode circuit; and a bridge molecule bridging across said sensor gap, connecting the source and drain electrodes; and a probe attached to the bridge molecule, wherein interaction of the probe with a ligand or binding partner is detectible by monitoring at least one parameter of the electrode circuit.

In accordance with some embodiments of the disclosure, a sensor includes a first contact coupled to a first electrode, a second contact coupled to a second electrode, a sensor gap defined between one of the first contact and the first electrode and one of the second contact and the second electrode, and a bridge molecule comprising a first end and a second end, wherein the bridge molecule is coupled to the first contact at the first end and coupled to the second contact at the second end. In accordance with various aspects of these embodiments, the bridge molecule is a peptide, protein, or derivative thereof. In accordance with additional aspects, the sensor optionally includes a third or gate electrode. In these cases, the gate electrode can be used to tune and/or activate the sensor device.

In another aspect, methods of molecular detection of a target that binds to the probe are provided herein. The method comprises, providing a sensor described herein, such as one comprising: a positive electrode; a negative electrode spaced apart from the positive electrode; a bridge-probe conjugate in electrical connection to positive and negative electrodes to form a conductive pathway between the positive and negative electrodes; initiating at least one of a voltage or a current through the circuit; exposing the circuit to a sample suspected of containing a target of interest, applied voltage on the primary electrodes, or voltage spectroscopy or sweeping applied to the primary electrodes; and measuring an electrical change in the circuit. In particular exemplary embodiments, the target of interest includes a virus such as COVID19, Hepatitis, HIV, Hepatovirus, and others, and methods of detection thereof are provided herein.

And, in accordance with further embodiments of the disclosure, a method of molecular detection of a target that binds to the probe is disclosed. The method comprises: providing a circuit further comprising a positive electrode; a negative electrode spaced apart from the positive electrode; and a bridge-probe conjugate connected to both the positive and negative electrodes to form a conductive pathway between the positive and negative electrodes; initiating at least one of a voltage or a current through the circuit; exposing the circuit to a solution containing target; and measuring electrical signals through the circuit as the probe binds to the target, wherein the electrical signals are processed to identify features that provide information on the underlying kinetics of the probe-target interaction.

### BRIEF DESCRIPTION OF THE DRAWINGS

FIG. 1 is a view illustrating the entire workflow for peptide bridges including design, expression and purification, helicity assessment, bioconjugation and biological assay.

FIG. 2 is a view illustrating the bridge design overview. The key elements include core alpha helix region, length of the alpha helix, anchor residues for metal binding and a single cysteine, lysine, or tyrosine residue practically in the middle of the whole sequence for probe bioconjugation. Figure discloses SEQ ID NOS 12 and 15-16, respectively, in order of appearance.

FIG. 3 is a view illustrating the representative sequence of bridge design centered around various metal electrodes. The key elements include anchor residues for metal binding and a single cysteine, lysine, or tyrosine residue near the middle of the whole sequence for probe bioconjugation.

FIG. 4 is a view illustrating Geobacter sulfurreducens Pilin-inspired constructs where the core alpha helical region is augmented for conductivity. The sequence is comprised of repeats of Pilin monomer aromatic rich region and EA3R (SEQ ID NO: 12) with and alternating Pilin regions with and without tyrosines.

FIG. 5 is a view illustrating the representative purification methods such as affinity, size exclusion, cation exchange, and hydrophobic interaction chromatographies, and subsequent qualitative analysis by gel electrophoresis.

FIG. 6 is a view illustrating size exclusion and reverse phase chromatography, with analytical gel electrophoresis after each step.

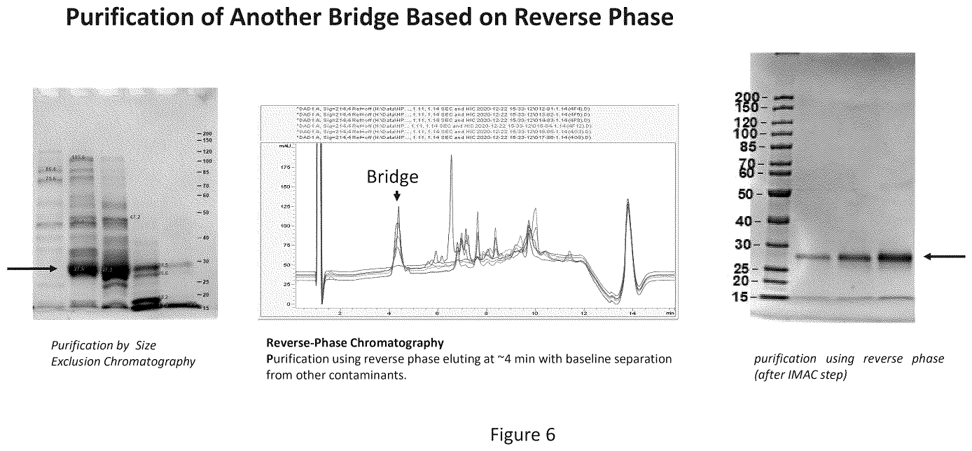

FIG. 7 is a view illustrating the representative circular dichroism (CD) spectrum measurements to estimate secondary structure of various peptide bridges.

FIG. 8 is a schematic showing the steps of probe bioconjugation to a bridge with a single central cysteine residue.

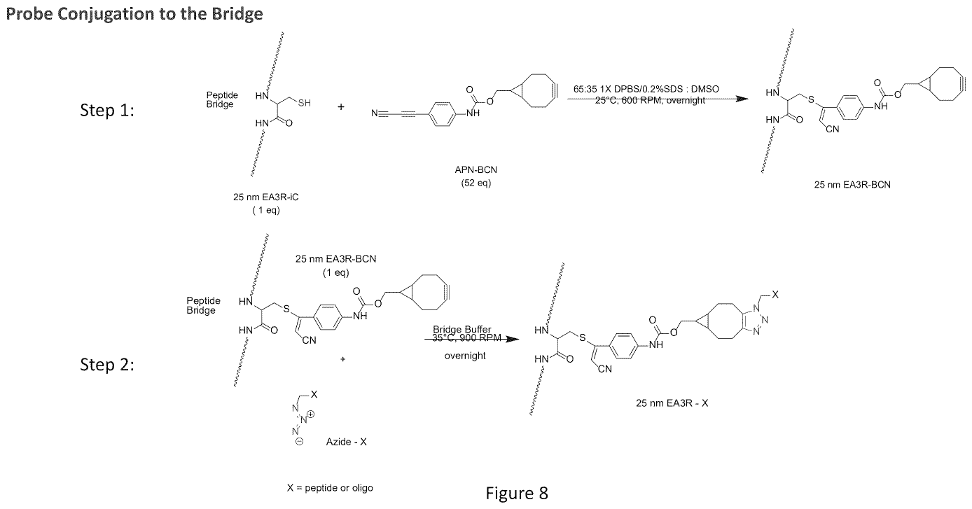

FIG. 9 is a schematic showing specific capping strategy at the terminus of peptide that can be utilized to improve bridge-electrode binding. This figure depicts partial anchoring strategy wherein a reactive handle X is installed via N-Capping with either vinyl boronate or activated ester on a bridge peptide.

FIG. 10 is a schematic showing specific capping strategy at the terminus of peptide that can be utilized to improve bridge-electrode binding. This figure shows dual reactive handle/anchor being installed via thiol bioconjugation post-N-capping on a bridge containing internal cysteines for conjugation.

FIG. 11 is a schematic showing various steps of probe bioconjugation targeting single central lysine residue.

FIG. 12 shows purification of a bridge conjugated with Hepatitis B surface Antigen aptamer Probe.

FIG. 13 shows isolation of a bridge conjugated with HIV-1 p24 antigen.

FIG. 14 shows purification of a bridge peptide conjugated with a DNA probe for the Hepatovirus A LTR region.

FIG. 15. In the scheme shown in FIG. 15, an aptamer specific to Hepatitis B surface Antigen is attached to the bridge. The binding response curve from various chips are also shown by plotting the fraction of the time bound versus the target antigen concentration.

FIG. 16 is a schematic showing HIV-1 p24 antigen attached to the bridge. The binding response curve from various chips are also shown by plotting the fraction of the time bound versus the antibody concentration.

FIG. 17 is a schematic showing a NAT specific DNA probe for Hepatovirus A LTR region is attached to the bridge. The binding response curve from various chips are also shown by plotting the fraction of the time bound versus the concentration of target made by Reverse-Transcriptase PCR from infected plasma.

FIG. 18 is a schematic that illustrates the general concept of engaging a binding probe molecule onto a molecular wire in an electronic circuit, to act as a sensor capable of detecting a binding event between the probe binding and its target, in accordance with various embodiments

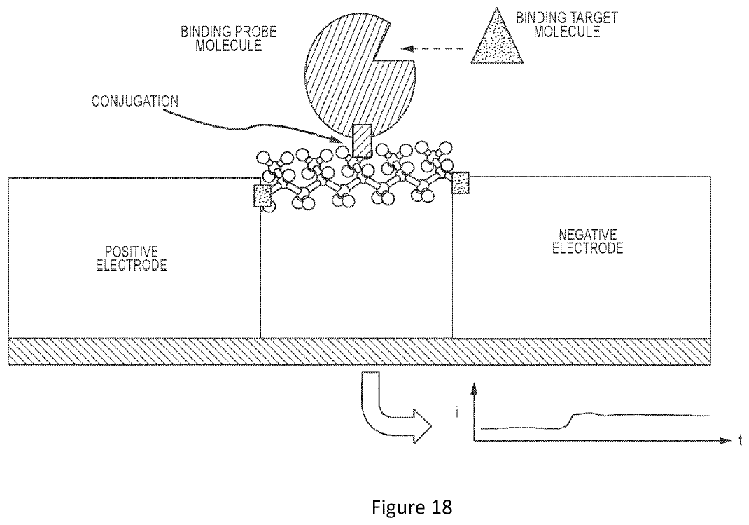

### DETAILED DESCRIPTION

Various aspects of the invention will now be described with reference to the following section which will be understood to be provided by way of illustration only and not to constitute a limitation on the scope of the invention.

### Definitions

As used herein, the term “bridge” or “bridge molecule” refers to a molecular wire or other electrically conducting molecule that may be used to make a conducting connection. Numerous bridges molecules are described in detail herein. Additionally, molecules that function as bridges include, but are not limited to, peptide alpha helices, polypeptides having particular amino acid sequences, graphene nanoribbons, pilin filaments or bacterial nanowires, double stranded DNA, other multichain proteins or conjugates of multiple single-chain proteins, antibodies, carbon nanotubes e.g., single-walled carbon nanotubes (CNTs, SWCNTs), semiconductor layers such as transition metal dichalcogenides (TMD) or other semiconductor nanoribbons or nanowires, or conducting polymers such as polythiophene, poly(3,4-ethylenedioxythiophene (PEDOT) or other synthetic electrically conducting polymers. Such molecules may include attachment groups, i.e., functionality that provide for specific attachment to, and/or self-assembly to, nanoelectrodes or contacts such as islands or deposits thereon.

As used herein, the term “electrode” means any structure that can act as an efficient source or sink of charge carriers. Most commonly these would be metal or semiconductor structures, such as those used in electronic circuits. A pair of spaced apart electrodes herein may comprise a source and drain electrode pair. In various embodiments of the present disclosure, a binding probe-based molecular circuit may further comprise a gate electrode. When present, a gate electrode is used to apply a voltage rather than transfer charge carriers. Thus it supports accumulation of charge carriers to produce a local electric field, but is not intended to pass current. A gate electrode will be electrically isolated from the primary conduction paths of the circuit by some form of insulating layer or material.

As used herein, the term “conjugation” means any of the wide variety of means of physically attaching one molecule to another, or to a surface or particle. Such methods typically involve forming covalent or non-covalent chemical bonds, but may also rely on protein-protein interactions, protein-metal interactions, or chemical or physical adsorption via intermolecular (Van der Waals) forces. There is a large variety of such methods know to those skilled in the art of conjugation chemistry. Common conjugation methods relevant to preferred embodiments herein include thiol-metal bonds, maleimide-cysteine bonds, material binding peptides such as gold binding peptides, and click chemistries.

In various embodiments of the present disclosure, a molecular sensor comprises a probe connected to both a positive and a negative electrode to complete a circuit. Interactions of the probe with ligand or binding partner are detectable as changes in the current or other electrical parameter measured across the circuit. The probe may be an enzyme or other protein conjugated to a bridge molecule as described. A second embodiment differs from the general concept of a molecular electronic circuit in that the enzyme is directly conjugated or “wired” to both the positive and negative electrodes rather than bonded to a molecular bridge molecule that spans the gap between the electrodes to complete a circuit.

In various aspects of the disclosure, at least one of a voltage or a current is initiated in a probe-based molecular circuit. When a target interacts with the probe, electrical changes in the circuit are sensed. These electrical changes, or informative electrical signals, may include current, voltage, impedance, conductivity, resistance, capacitance, or the like. In some examples, a voltage is initiated in the circuit and then changes in the current through the circuit are measured as substrates interact with the binding probe. In other examples, a current is initiated in the circuit, and changes to voltage in the circuit are measured as substrates interact with the enzyme. In other examples, impedance, conductivity, or resistance is measured. In examples wherein the circuit further comprises a gate electrode, such as positioned underneath the gap between the positive and negative electrodes, at least one of a voltage or current may be applied to the gate electrode, and voltage, current, impedance, conductivity, resistance, or other electrical change in the circuit may be measured as substrates interact with the binding probe. Suitable circuits are described in Applicant's prior related patent applications and patents, including U.S. Pat. Nos. 10,036,064, 10,508,296, 10,648,941, 10,584,410, 10,913,966, 11,143,617, and WO/2020/146823A9, all incorporated by reference herein in their entirety.

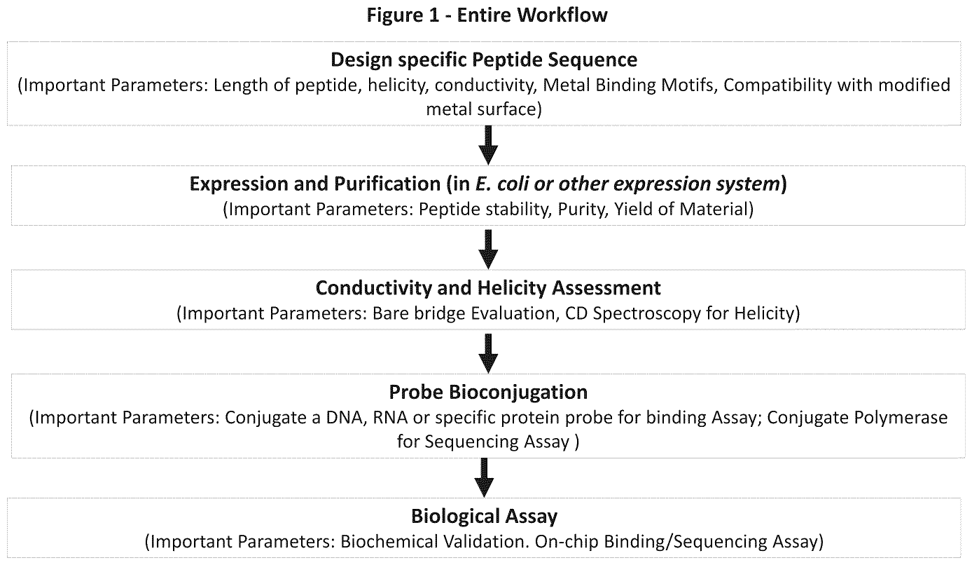

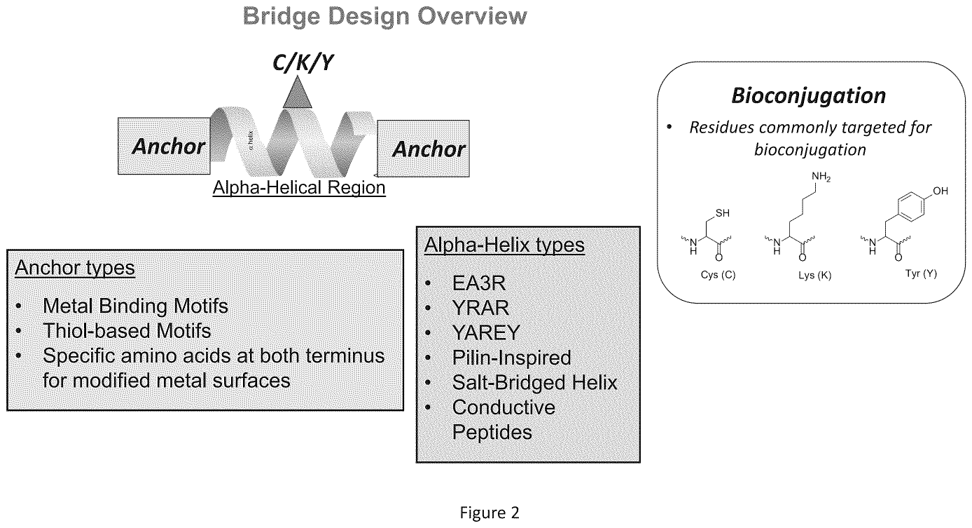

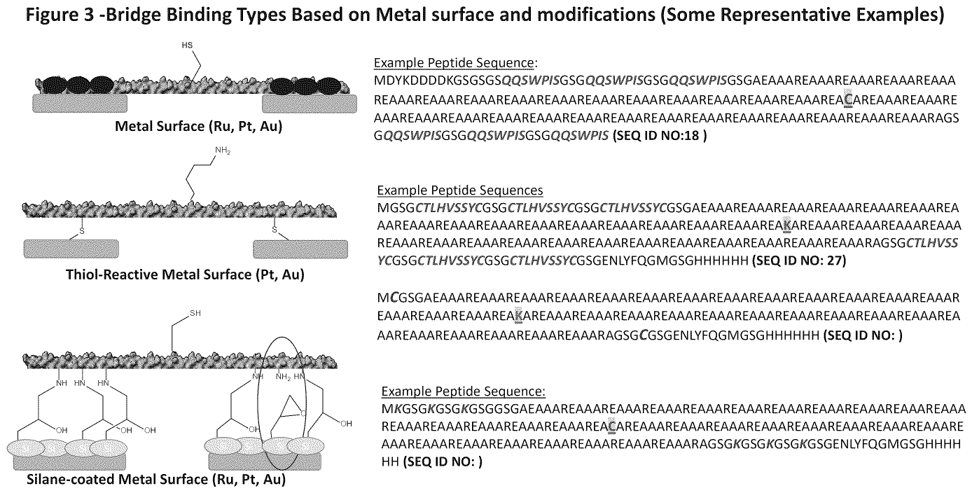

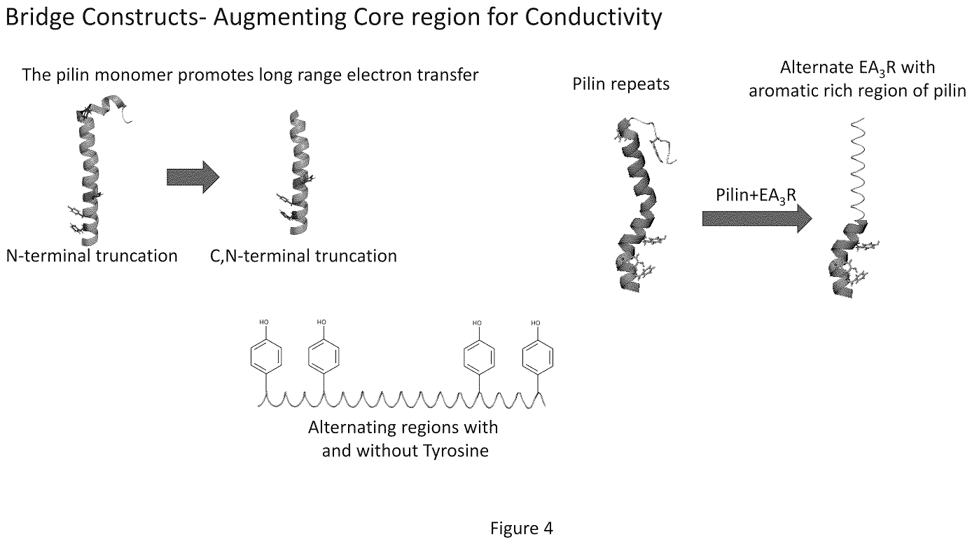

### Design and Sequence of Representative Peptide Bridges (FIG. 1-4)

Smaller peptides up to 10 nm in length that were less than 100 amino acids were chemically synthesized. Longer constructs ranging from 10-45 nm were designed in such a way that they could be recombinantly expressed in E. coli or other suitable expression systems.

The designed bridge peptides are alpha helix-forming sequences of various amino acids in length. Terminal tag sequences such as His-tag, FLAG-tag etc. are added to assist with the affinity chromatography purification. The tag can be cleavable, e.g., TEV cleavage sequence ENLYFQGM (SEQ ID NO: 1) can be added between bridge peptide and purification tag. Single or multiple repeats of metal-binding motifs at either one end or both ends of the alpha helical peptide were added that aids in attaching/binding of the peptide bridge to specific metal electrodes such as Ruthenium, Platinum, gold. The metal binding motifs contain one or a combination of single or multiple repeats of the following sequences: QQSWPIS (SEQ ID NO:2), CTLHVSSYC (SEQ ID NO:3), TLHVSSY (SEQ ID NO:4), KGSGKGSGK (SEQ ID NO:5), CCPGCC (SEQ ID NO:6), CPTSTGQA (SEQ ID NO:7), HFYWM (SEQ ID NO:8), HFYWMR (SEQ ID NO:9), PTSTGQA (SEQ ID NO:10), NFMSLPRLGHMH (SEQ ID NO:11), or any one of the preceding having one, two, three, four, or five amino acid deletions or conservative substitutions while retaining function. Other repeat motif used herein include EAAAR (SEQ ID NO:12), AREAL (SEQ ID NO: 14), YRAR (SEQ ID NO:15), YAREY (SEQ ID NO:16), or any one of the preceding having one amino acid deletion or conservative substitution while retaining function.

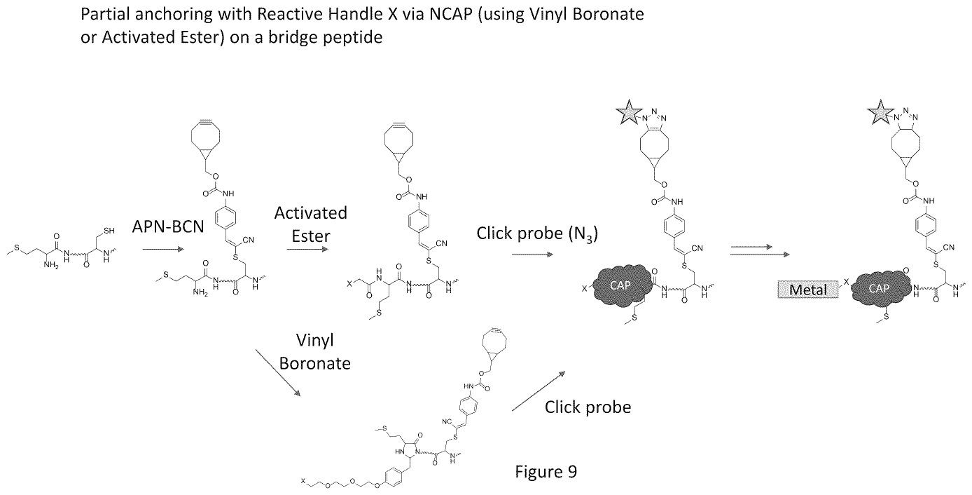

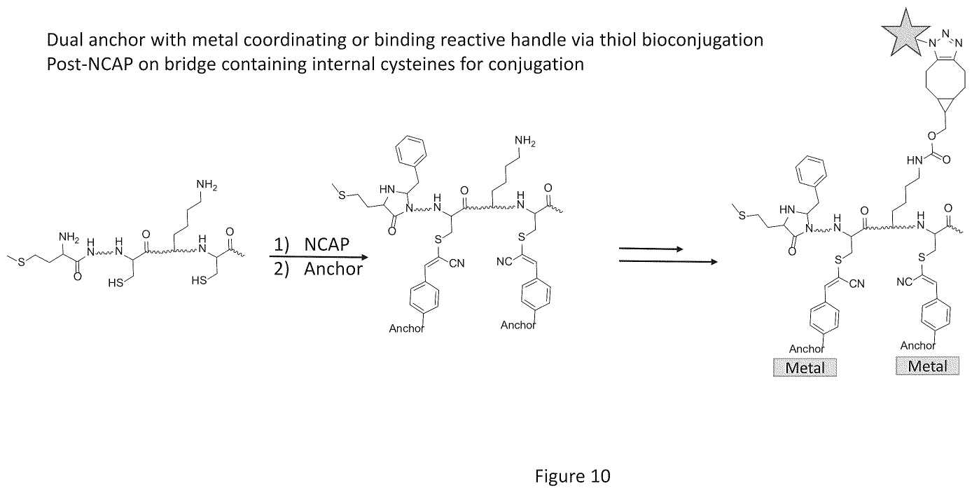

In another embodiment, specific capping strategy at the terminus of peptide can be utilized to improve bridge-electrode binding. For example, FIG. 9 depicts a strategy using vinyl boronate to CAP a peptide N-terminus. This can introduce a reactive moiety X with either vinyl boronate or activated ester on any bridge peptide. These reactive handles can be conjugated to sensor by reacting with specific click chemistry or epoxide, etc. or by coordinating the reactive moiety X to a functionalized surface. The functionalized surface can be achieved by utilizing silane, thiol, biotin-streptavidin, Ni-NTA-based chemistry onto various metal electrode surfaces such as ruthenium, platinum, or gold. In another embodiment, a dual reactive handle/anchor with metal coordinating or binding functionality can be installed via thiol bioconjugation post-NCAP on a bridge containing internal cysteines for conjugation (FIG. 10). The reactive moiety X can also be used to install compatible reactive sites for binding to or reacting with functionalized surfaces.

Sequence of an approximately 25 nm peptide bridge with an N-terminal FLAG tag, multiple repeats of core alpha helix comprising of EAAAR (SEQ ID NO: 12) sequence, 3 repeats of metal binding motifs preferably suitable for binding to Ru electrodes at two ends and a single cysteine residue in the middle of the entire peptide as the attachment point for probes using appropriate click chemistry such as alkyne/azide click chemistry.

(SEQ ID NO: 18)
MDYKDDDDKGSGSGSQQSWPISGSGQQSWPISGSGQQSWPISGSGAE
AAAREAAAREAAAREAAAREAAAREAAAREAAAREAAAREAAAREAA
AREAAAREAAAREAAAREAAAREAAAREAAAREACAREAAAREAAAR
EAAAREAAAREAAAREAAAREAAAREAAAREAAAREAAAREAAAREA
AAREAAAREAAAREAAAREAAARAGSGQQSWPISGSGQQSWPISGSG
QQSWPIS

Sequence of an approximately 40 nm peptide bridge with a C-terminal His tag, multiple repeats of core alpha helix comprising of EAAAR (SEQ ID NO: 12) sequence, 3 repeats of metal binding motif QQSWPIS (SEQ ID NO:2, shown in bold) preferably suitable for binding to Ru electrodes at two ends and a single cysteine residue in the middle of the entire peptide as the attachment point for probes using appropriate click chemistry such as alkyne/azide click chemistry.

(SEQ ID NO: 19)
MGSGQQSWPISGSGQQSWPISGSGQQSWPISGSGAEAAAREAAAREA
AAREAAAREAAAREAAAREAAAREAAAREAAAREAAAREAAAREAAA
REAAAREAAAREAAAREAAAREAAAREAAAREAAAREAAAREAAARE
AAAREAAAREAAAREAAAREAAAREACAREAAAREAAAREAAAREAA
AREAAAREAAAREAAAREAAAREAAAREAAAREAAAREAAAREAAAR
EAAAREAAAREAAAREAAAREAAAREAAAREAAAREAAAREAAAREA
AAREAAAREAAAREAAARAGSGQQSWPISGSGQQSWPISGSGQQSWP
ISGSGENLYFQGMGSGHHHHHH

Sequence of an approximately 25 nm peptide bridge with an N-terminal FLAG tag and a C-terminal His-tag, multiple repeats of core alpha helix comprising of EAAAR (SEQ ID NO:12) and EAYAR (SEQ ID NO: 13) sequences, repeats of metal binding motifs suitable for binding to Ru, Pt or Au electrodes at two ends and a single cysteine residue (underlined) in the middle of the entire peptide as the attachment point for probes using appropriate click chemistry such as alkyne/azide click chemistry.

(SEQ ID NO: 20)
MDYKDDDDKGSGENLYFQGMGSGKHFYWMGSGQQSWPISGSGHFYWM
GSGTLHVSSYGSGFYWMGSGYEAYAREAYAREAYAREAAAREAAARE
AAAREAYAREAYAREAYAREAAAREAAAREAAAREAYAREAYAREAY
AREAAAREAAAREACAREAAAREAAAREAYAREAYAREAYAREAAAR
EAAAREAAAREAYAREAYAREAYAREAAAREAAAREAAAREAYAREA
YAREAYARYGSGMHFYWGSGTLHVSSYGSGMHFYWGSGQQSWPISGS
GKMHFYWGSGHHHHHH

Sequence of an approximately 30 nm pilin-based peptide bridge with an N-terminal FLAG tag and a C-terminal His-tag, core with multiple repeats of modified pilin helix sequences, repeats of metal binding motifs suitable for binding to Ru, Pt and Au electrodes at two ends and a single cysteine residue in the middle of the entire peptide as the attachment point for probes using appropriate click chemistry such as alkyne/azide click chemistry.

(SEQ ID NO: 21)
MDYKDDDDKGSGENLYFQGMGSGKHFYWMGSGQQSWPISGSGHFY
WMGSGTLHVSSYGSGFYWMGSGAIPQFSAYRVKAYNSAASSDLRN
LKTALAIPQFSAYRVKAYNSAASSDLRNLKTALAIPQFSAYRVKA
YNSAASSDLRNLKTALAIPQFSAYRVKAYNSAASSDLRNLKTCLA
IPQFSAYRVKAYNSAASSDLRNLKTALAIPQFSAYRVKAYNSAAS
SDLRNLKTALAIPQFSAYRVKAYNSAASSDLRNLKTALAIPQFSA
YRVKAYNSAASSDLRNLKTALGSGMHFYWGSGTLHVSSYGSGMHF
YWGSGQQSWPISGSGKMHFYWGSGHHHHHH

Sequence of an approximately 25 nm pilin-based peptide bridge with a C-terminal His-tag, core comprising of alternating repeats of EAAAR (SEQ ID NO: 12) plus modified pilin helix sequences, repeats of metal binding motifs suitable for binding to Ru, Pt and Au electrodes at two ends and a single lysine residue in the middle of the entire peptide as the attachment point for probes using appropriate click chemistry such as alkyne/azide click chemistry.

(SEQ ID NO: 22)
ENLYFQGMGSGMGSGCTLHVSSYCGSGCTLHVSSYCGSGCTLHVS
SYCGSGAAIPQFSAYRVRAYNSEAAAREAAAREAAARAIPQFSAY
RVRAYNSEAAAREAAAREAAARAIPQFSAYRVRAYNSEAAAREAA
AREAKAREAAAREAAARAIPQFSAYRVRAYNSEAAAREAAAREAA
ARAIPQFSAYRVRAYNSEAAAREAAAREAAARAIPQFSAYRVRAY
NSAGSGCTLHVSSYCGSGCTLHVSSYCGSGCTLHVSSYCLEVLFQ
GPGSGHHHHHH

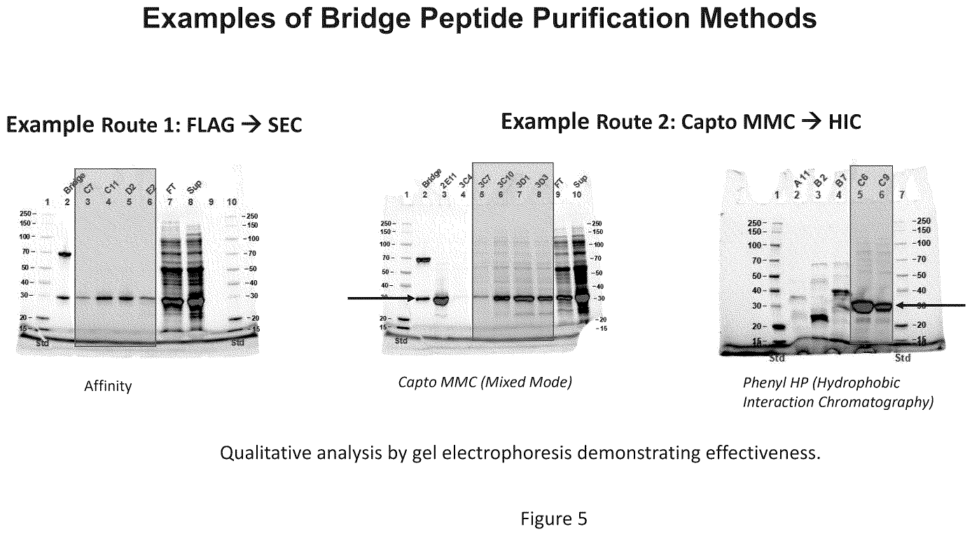

### Expression and Purification of Peptides (FIGS. 5 & 6)

pET vector was used for the expression of recombinant peptide gene in BL21(DE3) or BL21(DE3)pLysS E. coli strains. The first step in purification usually involved affinity chromatography step such as Ni-NTA or FLAG-resin based chromatography.

A second or third column-chromatography steps, such as cation exchange, hydrophobic interaction, reverse phase, or size exclusion chromatography were undertaken to achieve homogenous peptide with more than 90-95% purity.

### Conductivity and Helicity Assessment

Current-voltage (I/V-t) data was obtained on peptides by applying a set voltage across the chip and by recording the current. Applied voltage ranged between 0 V and 1.0 V with a 0.1 V step to yield the final IV curves. voltage range selection was chosen to be below 1.2 V to avoid electrochemical splitting of water. Helicity assessment was done using circular dichroism measurements

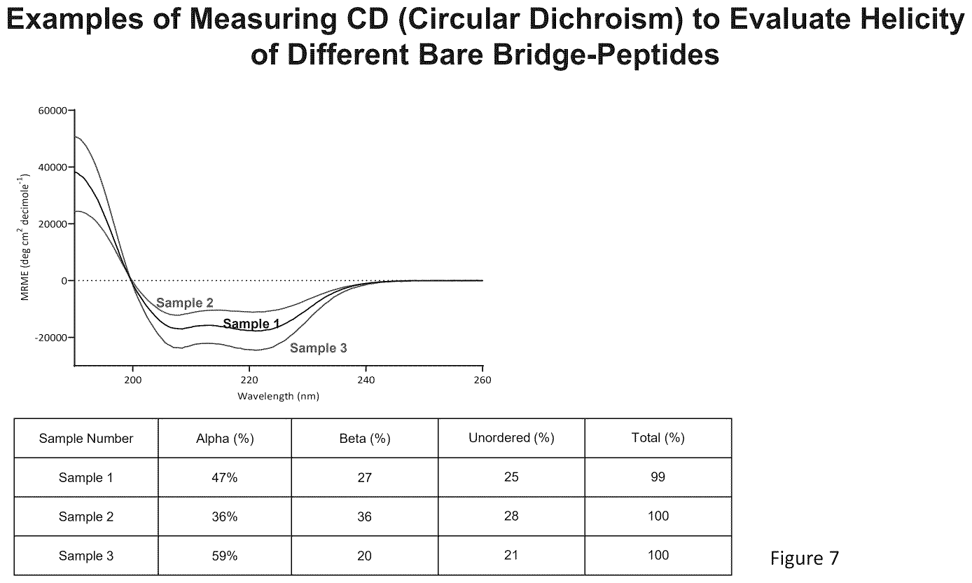

### Circular Dichroism (CD) Spectrometry (FIG. 7):

The CD measurements were performed in far-ultraviolet region of 250 nm to 190 nm on a Jasco J-720 spectropolarimeter (JASCO Inc.). For that, different peptides were added in cylinder type quartz cell having light path length of 0.1 cm. Three bridge-peptides were analyzed by far-ultraviolet (UV) circular dichroism (CD) to evaluate their secondary structure. The result showed about 59% alpha helical structure for bridge-peptide for approximately 25 nm peptide (Sample 3 peptide). Alpha helical structure of about 47% was observed for 40 nm peptide (Sample 1 peptide)

25 nm and 40 nm peptides have the same molar extinction coefficient, i.e., the same number of aromatic amino acids; however, 40 nm peptide has a greater number of core EAAAR repeats (SEQ ID NO: 12). Sample 2 is also about 25 nm in length; however, it contained multiple lysines at the 2-termini as metal binding motif. Sample 2 showed the alpha helical structure of 36%.

Sequence of Sample 1 peptide =
(SEQ ID NO: 23)
MGSGQQSWPISGSGQQSWPISGSGQQSWPISGSGAEAAAREAAAR
EAAAREAAAREAAAREAAAREAAAREAAAREAAAREAAAREAAAR
EAAAREAAAREAAAREAAAREAAAREAAAREAAAREAAAREAAAR
EAAAREAAAREAAAREAAAREAAAREAAAREACAREAAAREAAAR
EAAAREAAAREAAAREAAAREAAAREAAAREAAAREAAAREAAAR
EAAAREAAAREAAAREAAAREAAAREAAAREAAAREAAAREAAAR
EAAAREAAAREAAAREAAAREAAAREAAARAGSGQQSWPISGSGQ
QSWPISGSGQQSWPIS - GSG - ENLYFQGMGSGHHHHHH
Sequence of sample 2 peptide =
(SEQ ID NO: 24)
MWKGSGKGSGKGSGGSGAEAAAREAAAREAAAREAAAREAAAREA
AAREAAAREAAAREAAAREAAAREAAAREAAAREAAAREAAAREA
AAREAAAREACAREAAAREAAAREAAAREAAAREAAAREAAAREA
AAREAAAREAAAREAAAREAAAREAAAREAAAREAAAREAAAREA
AARAGSG-KGSGKGSGK GSG - WENLYFQGMGSGHHHHHH
Sequence of sample 3 peptide =
(SEQ ID NO: 25)
MDYKDDDDKGSGSGSQQSWPISGSGQQSWPISGSGQQSWPISGSG
AEAAAREAAAREAAAREAAAREAAAREAAAREAAAREAAAREAAA
REAAAREAAAREAAAREAAAREAAAREAAAREAAAREACAREAAA
REAAAREAAAREAAAREAAAREAAAREAAAREAAAREAAAREAAA
REAAAREAAAREAAAREAAAREAAAREAAARAGSGQQSWPISGSG
QQSWPISGSGQQSWPIS

### Bioconjugation Targeting Central Cysteine Residues (FIG. 9):

A bare bridge is a term that is used for the peptide bridge until a probe gets conjugated to it. In the first step, the bare peptide bridge is suspended in an appropriate aqueous buffer solution including stabilizers or detergents as needed. At room temperature, the cysteine selective clickable bioconjugation reagent is added as a solution in polar, water-miscible solvent. The reaction mixture is then incubated with mild agitation at temperatures ranging from 4 C to 30 C. Once sufficient time has elapsed, the crude reaction mixture is filtered to remove solids, and small molecule impurities are removed via diafiltration. The processed reaction mixture is then purified via preparative reverse phase high pressure liquid chromatography, generally eluting with a gradient of trifluoroacetic acid containing water and acetonitrile. The eluted fractions containing the product of interest are then diluted with water and frozen, followed by lyophilization. The lyophilized product is resuspended in an appropriate buffer solution, ensuring complete neutralization of residual acid. The purified product solution is quantified via gel densitometry and UV-Vis spectrophotometry and purity is assessed by SDS-PAGE and analytical HPLC.

In the subsequent step, the purified clickable protein intermediate solution is mixed with a solution of the probe of interest containing a compatible reactive functional group, generally but not limited to an azide. The probe-bridge reaction mixture is then incubated at an appropriate temperature, generally 4° C.-50° C. Once sufficient time has elapsed, the crude reaction mixture is concentrated via diafiltration and purified via Size Exclusion HPLC using an isocratic mobile phase containing appropriate buffer and stabilizers or detergents as needed. The eluted fractions containing the product of interest are then further concentrated and buffer exchanged into the desired final buffer composition via diafiltration. The purified product solution is quantified via gel densitometry and UV-Vis spectrophotometry and purity is assessed by SDS-PAGE and analytical HPLC.

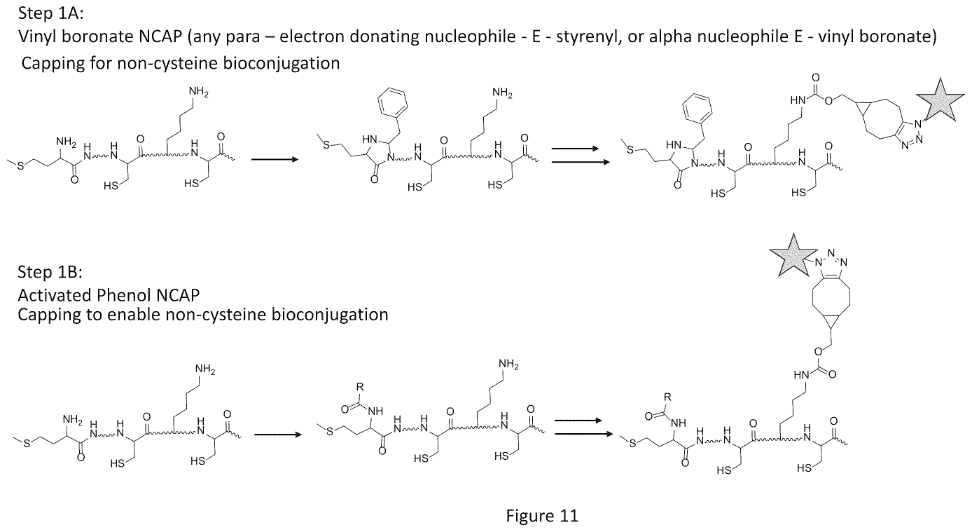

FIG. 9: A synthetic scheme demonstrating the asymmetric incorporation of a direct or indirect binding moieties at the N-Terminus of the peptide bridge starting with either activated phenol (see STEP 1B of FIG. 11) or vinyl boronate (see Step 1A of FIG. 11).

FIG. 10: A synthetic scheme demonstrating the multi-incorporation of direct or indirect binding moieties embedded within the backbone of the peptide bridge starting with either activated phenol (see STEP 1B of FIG. 11) or vinyl boronate (see Step 1A of FIG. 11) capping, and residue selective anchor incorporation.

### Bioconjugation Targeting Central Lysine Residues (FIG. 11):

In the first step, the bare peptide bridge is buffer exchanged into a neutral non-amine buffer solution. Subsequently, a large excess of sodium ascorbate is added as freshly prepared aqueous solution, followed by the addition of excess of vinyl boronate. The resulting mixture is vortexed, centrifuged and allowed to incubate for several hours to overnight at 4° C. or room temperature, as appropriate for the bridge being used. The small molecule reactants are removed via centrifugal diafiltration with an appropriately sized membrane filter and optionally purified via suitable chromatographic steps to obtain capped-peptide bridge intermediate.

Alternatively, the capped-peptide bridge intermediate can be obtained by buffer exchanging the bare protein bridge into a neutral non-amine buffer solution. Afterwards, the activated phenol is added in an anhydrous polar water miscible solvent. The resulting mixture is vortexed, centrifuged and allowed to incubate for several hours to overnight at 4 C or room temperature, as appropriate for the bridge being used. The small molecule reactants are removed via centrifugal diafiltration with an appropriately sized membrane filter and optionally purified via suitable chromatographic steps.

In a second step, the capped-peptide bridge is suspended in an appropriate basic aqueous buffer solution including stabilizers or detergents as needed. At room temperature, the lysine selective clickable bioconjugation reagent is added as a solution in polar, water miscible solvent. The reaction mixture is then incubated with mild agitation at temperatures ranging from 4° C. to 30° C. Once sufficient time has elapsed, the crude reaction mixture is filtered to remove solids, and small molecule impurities are removed via diafiltration. Optionally, the processed reaction mixture is then purified via preparative reverse phase high pressure liquid chromatography, generally eluting with a gradient of trifluoroacetic acid containing water and acetonitrile. The eluted fractions containing the product of interest are then diluted with water and frozen, followed by lyophilization. The lyophilized product is resuspended in an appropriate buffer solution, ensuring complete neutralization of residual acid. The crude or purified product solution is quantified via gel densitometry and UV-Vis spectrophotometry and purity is assessed by SDS-PAGE and analytical HPLC.

In the subsequent step, the crude or purified clickable protein intermediate solution is mixed with a solution of the probe of interest containing a compatible reactive functional group, generally but not limited to an azide. The probe-bridge reaction mixture is then incubated at an appropriate temperature, generally 4° C.-50° C. Once sufficient time has elapsed, the crude reaction mixture is concentrated via diafiltration and purified via Size Exclusion HPLC using an isocratic mobile phase containing appropriate buffer such as PBS and stabilizers or detergents as needed. The eluted fractions containing the product of interest are then further concentrated and buffer exchanged into the desired final buffer composition via diafiltration. The purified product solution is quantified via gel densitometry and UV-Vis spectrophotometry and purity is assessed by SDS-PAGE and analytical HPLC.

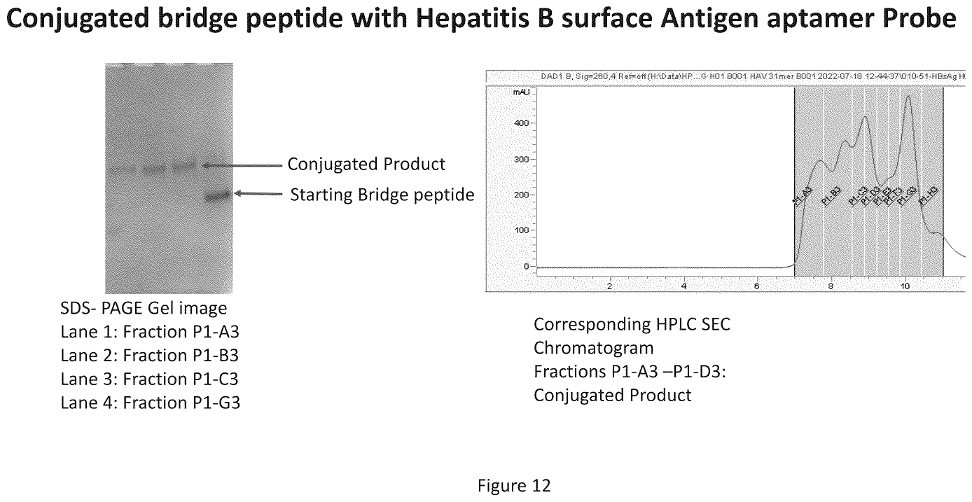

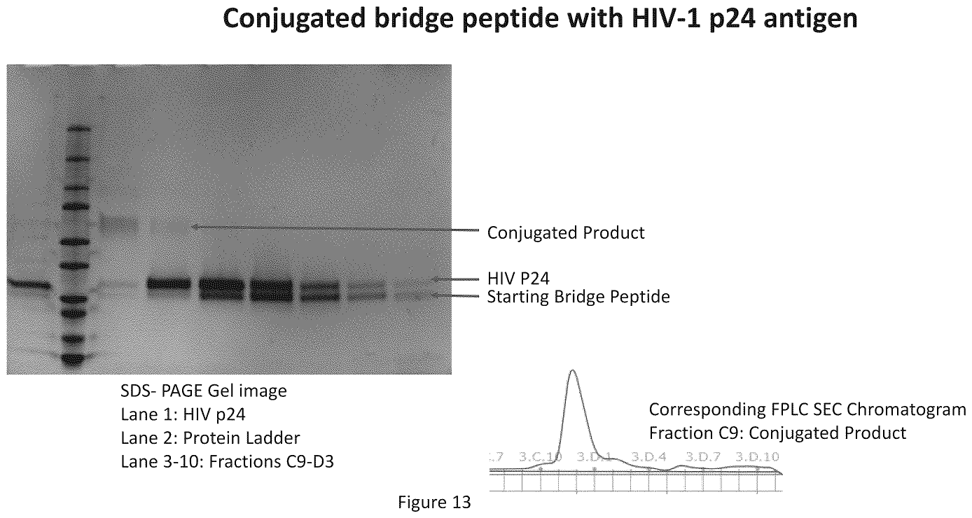

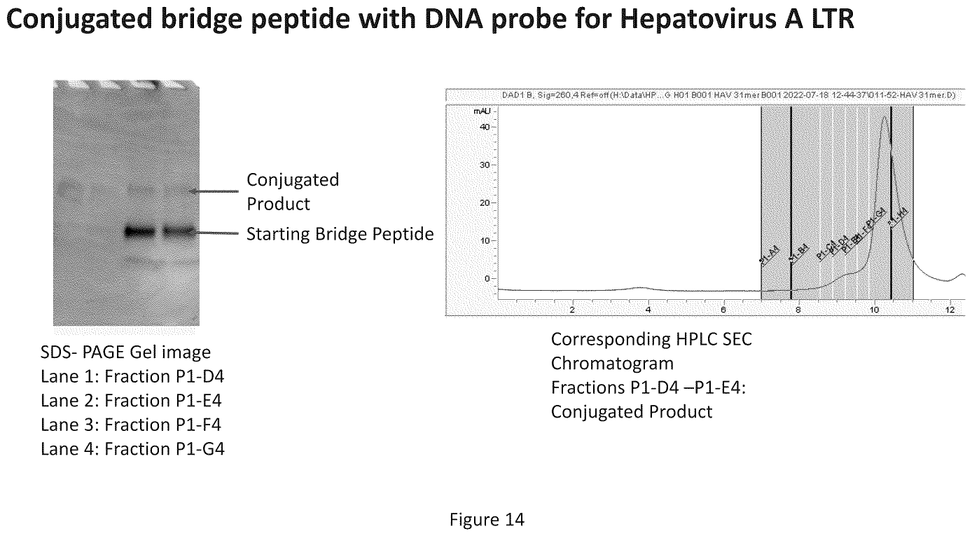

### Binding Assays (FIG. 12-17):

To show versatility of peptide bridges, we have demonstrated a variety of model binding assays, including DNA-DNA hybridization, DNA-protein interaction, and protein-protein interaction.

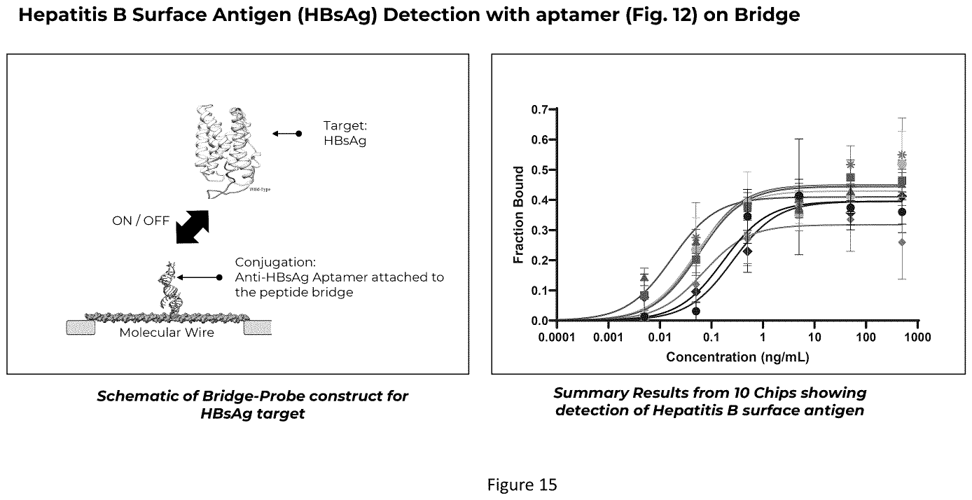

In the scheme shown in FIG. 15, where an aptamer is attached to the bridge, we show a nucleic acid-protein binding. The probe on the peptide bridge is a DNA aptamer (a single-stranded DNA, of about 70 nt in length) having an affinity for Hepatitis B Surface antigen. In the presence of the target Hepatis B surface antigen protein, the sensor current exhibits pulses corresponding to individual aptamer-protein binding events. The rate of pulse detection (and the fraction of time in the bound state) increases with higher target molecule concentration. Plotting the fraction of the time bound versus the target concentration produces a classical binding response curve.

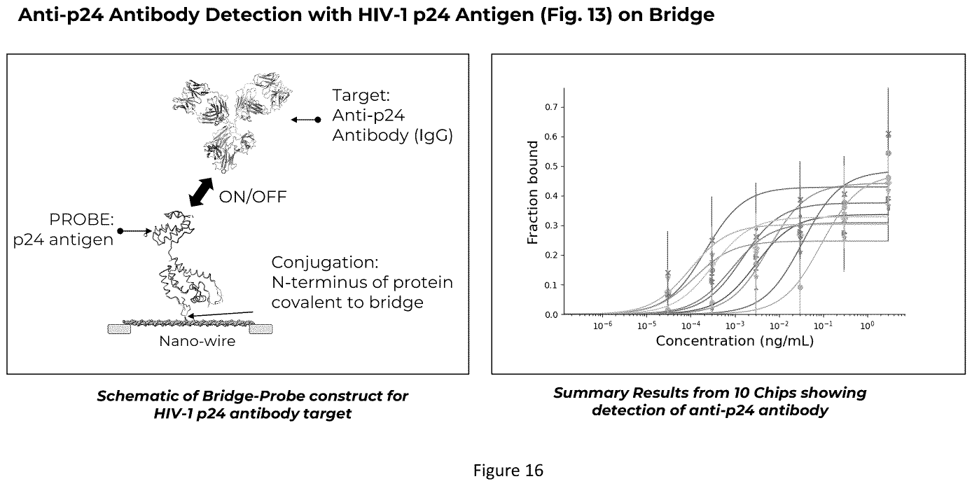

In the scheme shown in FIG. 16, where a protein or antigen is attached to the bridge, we show a protein-protein binding. The probe on the peptide bridge is HIV p24 antigen having an affinity for anti-p24 antibodies. In the presence of the target anti-p24 antibodies, the sensor current exhibits pulses corresponding to antigen-antibody binding events. Plotting the fraction of the time bound versus the target concentration produces a typical binding response curve for this antigen-antibody interaction.

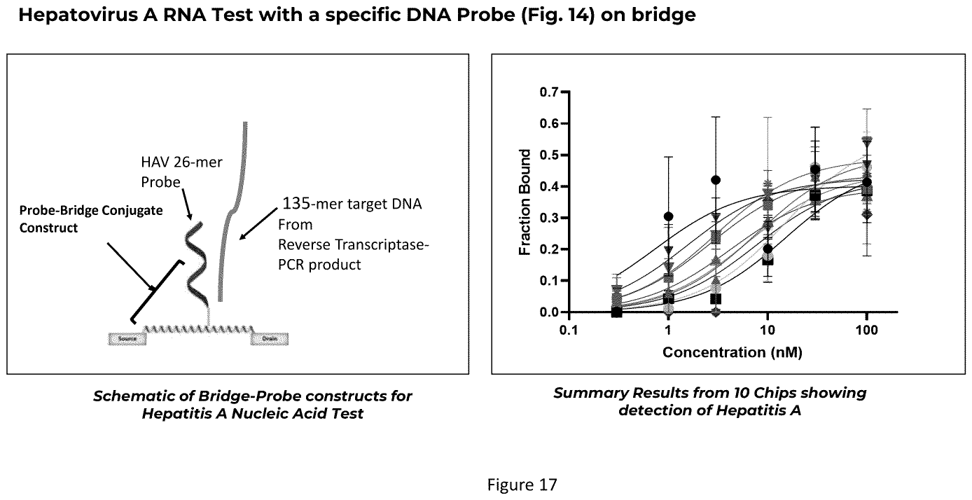

In the scheme shown in FIG. 17, where a Hepatitis A NAT probe is attached to the bridge, we show a DNA-DNA binding. The probe on the peptide bridge is 26-mer DNA from the LTR region of Hepatitis A virus. In the presence of the complementary PCR target, the sensor current exhibits pulses corresponding to antigen-antibody binding events. Plotting the fraction of the time bound versus the target concentration produces a typical binding response curve for this DNA-DNA hybridization.

### Additional Sequences of the Invention

With reference to the binding motif sequences below, the literature references below were used for some sequences. Chiu, C. Y et al. Size-controlled synthesis of Pd nanocrystals using a specific multifunctional peptide. Nanoscale 2010, 2, 927-930; Pacardo, D. B. et al. Biomimetic synthesis of Pd nanocatalysts for the Stille coupling reaction. ACS Nano 2009, 3, 1288-1296; and Seker U. O. S. et. al. Material Binding Peptides for Nanotechnology. Molecules 2011, 16, 1426-1451.

Binding Motifs:
(SEQ ID NO: 2)
QQSWPIS
(SEQ ID NO: 3)
CTLHVSSYC
(SEQ ID NO: 4)
TLHVSSY
(SEQ ID NO: 5)
KGSGKGSGK
(SEQ ID NO: 6)
CCPGCC
(SEQ ID NO: 7)
CPTSTGQA
(SEQ ID NO: 8)
HFYWM
(SEQ ID NO: 9)
HFYWMR
(SEQ ID NO: 10)
PTSTGQA
(SEQ ID NO: 11)
NFMSLPRLGHMH
Repeat Motifs
(SEQ ID NO: 12)
EAAAR
(SEQ ID NO: 13)
EAYAR
(SEQ ID NO: 14)
AREAL
(SEQ ID NO: 15)
YRAR
(SEQ ID NO: 16)
YAREY
Alternative bridge molecule amino acid sequences
used in embodiments of the invention are
listed below
(SEQ ID NO: 26)
QQSWPISGSGQQSWPISGSGQQSWPISGSGAEAAAREAAAREAAAREAAAREAA
AREAAAREAAAREAAAREAAAREAAAREAAAREAAAREAAAREAAAREAAAREAAAREACA
REAAAREAAAREAAAREAAAREAAAREAAAREAAAREAAAREAAAREAAAREAAAREAAAR
EAAAREAAAREAAAREAAARAGSGQQSWPISGSGQQSWPISGSGQQSWPIS -
GSGENLYFQGMGSGHHHHHH
(SEQ ID NO: 27)
MGSGCTLHVSSYCGSGCTLHVSSYCGSGCTLHVSSYCGSGAEAAAREAAAREA
AAREAAAREAAAREAAAREAAAREAAAREAAAREAAAREAAAREAAAREAAAREAAAREAA
AREAAAREAKAREAAAREAAAREAAAREAAAREAAAREAAAREAAAREAAAREAAAREAAA
REAAAREAAAREAAAREAAAREAAAREAAARAGSGCTLHVSSYCGSGCTLHVSSYCGSGCTLH
VSSYCGSGENLYFQGMGSGHHHHHH
(SEQ ID NO: 28)
MGSGQQSWPISGSGQQSWPISGSGQQSWPISGSGAEAAAREAAAREAAAREAAA
REAAAREAAAREAAAREAAAREAAAREAAAREAAAREAAAREAAAREAAAREAAAREAAAR
EAAAREAAAREAAAREAAAREACAREAAAREAAAREAAAREAAAREAAAREAAAREAAARE
AAAREAAAREAAAREAAAREAAAREAAAREAAAREAAAREAAAREAAAREAAAREAAAREA
AARAGSGQQSWPISGSGQQSWPISGSGQQSWPISGSGENLYFQGMGSGHHHHHH
(SEQ ID NO: 29)
MGSGCTLHVSSYCGSGCTLHVSSYCGSGCTLHVSSYCGSGAEAAAREAAAREA
AAREAAAREAAAREAAAREAAAREAAAREAAAREAAAREAAAREAAAREAAAREAAAREAA
AREAAAREAAAREAAAREAAAREAAAREAAAREAAAREAAAREAAAREAAAREAAAREAKA
REAAAREAAAREAAAREAAAREAAAREAAAREAAAREAAAREAAAREAAAREAAAREAAAR
EAAAREAAAREAAAREAAAREAAAREAAAREAAAREAAAREAAAREAAAREAAAREAAARE
AAAREAAARAGSGCTLHVSSYCGSGCTLHVSSYCGSGCTLHVSSYCGSGENLYFQGMGSGHHH
HHH
(SEQ ID NO: 30)
MGSGTLHVSSYGSGTLHVSSYGSGTLHVSSYGSGAEAAAREAAAREAAAREAA
AREAAAREAAAREAAAREAAAREAAAREAAAREAAAREAAAREAAAREAAAREAAAREAAA
REAAAREAAAREAAAREAAAREACAREAAAREAAAREAAAREAAAREAAAREAAAREAAAR
EAAAREAAAREAAAREAAAREAAAREAAAREAAAREAAAREAAAREAAAREAAAREAAARE
AAARAGSGTLHVSSYGSGTLHVSSYGSGTLHVSSYGSGENLYFQGMGSGHHHHHH
(SEQ ID NO: 31)
MGSGTLHVSSYGSGTLHVSSYGSGTLHVSSYGSGAEAAAREAAAREAAAREAA
AREAAAREAAAREAAAREAAAREAAAREAAAREAAAREAAAREAAAREAAAREAAAREAAA
REAAAREAAAREAAAREAAAREAAAREAAAREAAAREAAAREAAAREAAAREACAREAAAR
EAAAREAAAREAAAREAAAREAAAREAAAREAAAREAAAREAAAREAAAREAAAREAAARE
AAAREAAAREAAAREAAAREAAAREAAAREAAAREAAAREAAAREAAAREAAAREAAAREA
AARAGSGTLHVSSYGSGTLHVSSYGSGTLHVSSYGSGENLYFQGMGSGHHHHHH
(SEQ ID NO: 32)
MCGSGAEAAAREAAAREAAAREAAAREAAAREAAAREAAAREAAAREAAARE
AAAREAAAREAAAREAAAREAAAREAAAREAAAREAAAREAAAREAAAREAAAREAAAREA
AAREAKAREAAAREAAAREAAAREAAAREAAAREAAAREAAAREAAAREAAAREAAAREAA
AREAAAREAAAREAAAREAAAREAAAREAAAREAAAREAAAREAAAREAAAREAAARAGSG
CGSGENLYFQGMGSGHHHHHH
(SEQ ID NO: 33)
MKGSGKGSGKGSGAEAAAREAAAREAAAREAAAREAAAREAAAREAAAREAA
AREAAAREAAAREAAAREAAAREAAAREAAAREAAAREAAAREAAAREAAAREAAAREAAA
REAAAREAAAREACAREAAAREAAAREAAAREAAAREAAAREAAAREAAAREAAAREAAAR
EAAAREAAAREAAAREAAAREAAAREAAAREAAAREAAAREAAAREAAAREAAAREAAARE
AAARAGSG-KGSGKGSGK GSGENLYFQGMGSGHHHHHH
(SEQ ID NO: 34)
MCTLHVSSYCGSGAEAAAREAAAREAAAREAAAREAAAREAAAREAAAREAA
AREAAAREAAAREAAAREAAAREAAAREAAAREAAAREAAAREAKAREAAAREAAAREAAA
REAAAREAAAREAAAREAAAREAAAREAAAREAAAREAAAREAAAREAAAREAAAREAAAR
EAAARAGSG-CTLHVSSYC GSGENLYFQGMGSGHHHHHH
(SEQ ID NO: 35)
QQSWPISGSGQQSWPISGSGQQSWPISGSGAEAAAREAAAREAAAREAAAREAA
AREAAAREAAAREAAAREAAAREAAAREAAAREAAAREAAAREAAAREAAAREAAAREAKA
REAAAREAAAREAAAREAAAREAAAREAAAREAAAREAAAREAAAREAAAREAAAREAAAR
EAAAREAAAREAAAREAAARAGSGQQSWPISGSGQQSWPISGSGQQSWPISGSGENLYFQGMGS
GHHHHHH
(SEQ ID NO: 36)
MGSGCCPGCCGSGQQSWPISGSGAEAAAREAAAREAAAREAAAREAAAREAAA
REAAAREAAAREAAAREAAAREAAAREAAAREAAAREAAAREAAAREAAAREAAAREAAAR
EAAAREAAAREAKAREAAAREAAAREAAAREAAAREAAAREAAAREAAAREAAAREAAARE
AAAREAAAREAAAREAAAREAAAREAAAREAAAREAAAREAAAREAAAREAAARAGSGQQS
WPISGSGCCPGCCGSGENLYFQGMGSGHHHHHH
(SEQ ID NO: 37)
MGSGCCPGCCGSGTLHVSSYWGSGAEAAAREAAAREAAAREAAAREAAAREA
AAREAAAREAAAREAAAREAAAREAAAREAAAREAAAREAAAREAAAREAAAREAAAREAA
AREAAAREAAAREAKAREAAAREAAAREAAAREAAAREAAAREAAAREAAAREAAAREAAA
REAAAREAAAREAAAREAAAREAAAREAAAREAAAREAAAREAAAREAAAREAAARAGSG
TLHVSSYW GSG CCPGCCGSG - ENLYFQGMGSGHHHHHH
(SEQ ID NO: 38)
MWCGSGAEAAAREAAAREAAAREAAAREAAAREAAAREAAAREAAAREAAA
REAAAREAAAREAAAREAAAREAAAREAAAREAAAREAKAREAAAREAAAREAAAREAAAR
EAAAREAAAREAAAREAAAREAAAREAAAREAAAREAAAREAAAREAAAREAAAREAAARA
GSGCGSGWENLYFQGMGSGHHHHHH
(SEQ ID NO: 39)
MWCGSGAEAAAREAAAREAAAREAAAREAAAREAAAREAAAREAAAREAAA
REAAAREAAAREAAAREAAAREAAAREAAAREAAAREAAAREAAAREAAAREAAAREAAAR
EAAAREAKAREAAAREAAAREAAAREAAAREAAAREAAAREAAAREAAAREAAAREAAARE
AAAREAAAREAAAREAAAREAAAREAAAREAAAREAAAREAAAREAAAREAAAREAAARAG
SGCGSGWENLYFQGMGSGHHHHHH
(SEQ ID NO: 40)
MWKGSGKGSGKGSGAEAAAREAAAREAAAREAAAREAAAREAAAREAAARE
AAAREAAAREAAAREAAAREAAAREAAAREAAAREAAAREAAAREAAAREAAAREAAAREA
AAREAAAREAAAREACAREAAAREAAAREAAAREAAAREAAAREAAAREAAAREAAAREAA
AREAAAREAAAREAAAREAAAREAAAREAAAREAAAREAAAREAAAREAAAREAAAREAAA
REAAARAGSG-KGSGKGSGK GSG - WENLYFQGMGSGHHHHHH
(SEQ ID NO: 41)
WSHPQFEKENLYFQGMGSGCTLHVSSYCGSGQQSWPISGSGCTLHVSSYCGSGQ
QSWPISGSGAEAAAREAAAREAAAREAAAREAAAREAAAREAAAREAAAREAAAREAAAREA
AAREAAAREAAAREAAAREAAAREAAAREAAAREAAAREAAAREAAAREAAAREAAAREAA
AREAAAREAAAREAAAREAKAREAAAREAAAREAAAREAAAREAAAREAAAREAAAREAAA
REAAAREAAAREAAAREAAAREAAAREAAAREAAAREAAAREAAAREAAAREAAAREAAAR
EAAAREAAAREAAAREAAAREAAAREAAARAGSGQQSWPISGSGCTLHVSSYCGSGCTLHVSS
YCGSGQQSWPIS - GSGHHHHHH
(SEQ ID NO: 42)
WSHPQFEKENLYFQGMGSGCCPGCCGSGQQSWPISGSGQQSWPISGSGAEAAAR
EAAAREAAAREAAAREAAAREAAAREAAAREAAAREAAAREAAAREAAAREAAAREAAARE
AAAREAAAREAAAREAKAREAAAREAAAREAAAREAAAREAAAREAAAREAAAREAAAREA
AAREAAAREAAAREAAAREAAAREAAAREAAAREAAARAGSGQQSWPIS GSG
QQSWPISGSGCCPGCCGSGHHHHHH
(SEQ ID NO: 43)
WSHPQFEKENLYFQGMGSGCCPGCCGSGQQSWPISGSGQQSWPISGSGAEAAAR
EAAAREAAAREAAAREAAAREAAAREAAAREAAAREAAAREAAAREAAAREAAAREAAARE
AAAREAAAREAAAREAYAREAAAREAAAREAAAREAAAREAAAREAAAREAAAREAAAREA
AAREAAAREAAAREAAAREAAAREAAAREAAAREAAARAGSGQQSWPISGSGQQSWPISGSGC
CPGCCGSGHHHHHH
(SEQ ID NO: 44)
MDYKDDDDKGSGENLYFQGMGSGKHFYWMGSGQQSWPISGSGHFYWMGSGT
LHVSSYGSGHFYWMGSGAEAAAREAAAREAAAREAAAREAAAREAAAREAAAREAAAREAA
AREAAAREAAAREAAAREAAAREAAAREAAAREAAAREACAREAAAREAAAREAAAREAAA
REAAAREAAAREAAAREAAAREAAAREAAAREAAAREAAAREAAAREAAAREAAAREAAAR
AGSGMHFYWGSGTLHVSSYGSGMHFYWGSGQQSWPISGSGKMHFYWGSGHHHHHH
(SEQ ID NO: 45)
MEAAAR MAAAF YAAWR EAMAR YEAFR
GSGAEAAAREAAAREAAAREAAAREAAAREAAAREAAAREAAAREAAAREAAAREAAAREAAAR
EAAAREAAAREAAAREAAAREACAREAAAREAAAREAAAREAAAREAAAREAAAREAAAREAAA
REAAAREAAAREAAAREAAAREAAAREAAAREAAAREAARAGSGEAAARMAAAFYAAWREAMA
RYEAFRDYKDDDDKGSGENLYFQGMGSGHHHHHH
(SEQ ID NO: 46)
MDYKDDDDKGSGENLYFQGMGSGKQQSWPISGSGMQQSWPISGSGMQQSWPIS
GSGAIPQFSAYRVKAYNSAASSDLRNLKTALAIPQFSAYRVKAYNSAASSDLRNLKTALAIPQFS
AYRVKAYNSAASSDLRNLKTALAIPQFSAYRVKAYNSAASSDLRNLKTALAIPQFSAYRVKAYN
SAASSDLRNLKTALAIPQFSAYRVKAYNSAASSDLRNLKTCLAIPQFSAYRVKAYNSAASSDLRN
LKTALAIPQFSAYRVKAYNSAASSDLRNLKTALAIPQFSAYRVKAYNSAASSDLRNLKTALAIPQ
FSAYRVKAYNSAASSDLRNLKTALAIPQFSAYRVKAYNSAASSDLRNLKTALAIPQFSAYRVKA
YNSAASSDLRNLKTALGSGQQSWPISGSGMQQSWPISGSGKQQSWPISMGSGHHHHHH
SMGSGFTLHVSSYFMGSGFTLHVSSYFMGSGKQQSWPISGSGHHHHHH
(SEQ ID NO: 47)
MDYKDDDDKGSGENLYFQGMGSGKFTLHVSSYFMGSGQQSWPISGSGFTLHVS
SYFGSGMQQSWPISFEAYAREYREAYAREYAAREAAAREAAAREAYAREYREAYAREYAAAR
EAAAREAAAREAYAREYREAYAREYAAAREACAREAAAREAYAREYREAYAREYAAAREAA
AREAAAREAYAREYREAYAREYAAAREAAAREAAAREAYAREYREAYAREY AFGSGQQSWPI
(SEQ ID NO: 48)
MDYKDDDDKGSGENLYFQGMGSGKFTLHVSSYFMGSGQQSWPISGSGFTLHVS
SYFGSGMQQSWPISFSYRARAYRARAYRARAAADAAADDAAADDAYRARAYRARAYRARAA
ADAAADDAAADDAYRARAYRARAYRARAAADAACDDAAADDAYRARAYRARAYRARAAA
DAAADDAAADDAYRARAYRARAYRARAAADAAADDAAADDAYRARAYRARAYRARSFGSG
QQSWPISMGSGFTLHVSSYFMGSGFTLHVSSYFMGSGKQQSWPISGSGHHHHHH
(SEQ ID NO: 49)
MDYKDDDDKGSGENLYFQGMGSGKHFYWMGSGQQSWPISGSGHFYWMGSGT
LHVSSYGSGHFYWMGSGFSYRARAYRARAYRARAAAARRRREEEEAAAAYRARAYRARAYR
ARAAAARRRREEEEAAAAYRARAYRARAYRARAAAARRRCEEEAAAAYRARAYRARAYRAR
AAAARRRREEEEAAAAYRARAYRARAYRARAAAARRRREEEEAAAAYRARAYRARAYRARSF
GSGMHFYWGSGTLHVSSYGSGMHFYWGSGQQSWPISGSGKMHFYWGSGHHHHHH
(SEQ ID NO: 50)
MDYKDDDDKGSGENLYFQSGSGQQSWPISGSGQQSWPISGSGQQSWPISGSGAE
AAAREAY AREAAAREAAAREAAAREAAAREAAAREAAAREAAAREAAAREAAAREAAAREA
AAREAAAREAAAREAAAREACAREAAAREAAAREAAAREAAAREAAAREAAAREAAAREAA
AREAAAREAKAREAAAREAAAREAAAREAAAREAAAREAAARAGSGQQSWPISGSGQQSWPIS
GSGQQSWPISGSGLEVLFQGPKHHHHHH
(SEQ ID NO: 51)
MDYKDDDDKGSGENLYFQGMGSGKHFYWMGSGQQSWPISGSGHFYWMGSGT
LHVSSYGSGFYWMGSGYEAYAREAYAREAYAREAAAREAAAREAAAREAYAREAYAREAYA
REAAAREAAAREAAAREAYAREAYAREAYAREAAAREAAAREACAREAAAREAAAREAYAR
EAYAREAYAREAAAREAAAREAAAREAYAREAYAREAYAREAAAREAAAREAAAREAYARE
AYAREAYARYGSGMHFYWGSGTLHVSSYGSGMHFYWGSGQQSWPISGSGKMHFYWGSGHHH
HHH
(SEQ ID NO: 52)
ENLYFQGMGSGMGSGCTLHVSSYCGSGCTLHVSSYCGSGCTLHVSSYCGSGAY
EAYAREAYAREAYAREAAAREAAAREAAAREAYAREAYAREAYAREAAAREAAAREAAARE
AYAREAYAREAYAREAAAREAAAREAKAREAAAREAAAREAYAREAYAREAYAREAAAREA
AAREAAAREAYAREAYAREAY AREAAAREAAAREAAAREAYAREAYAREAYARYAGSGCTL
HVSSYCGSGCTLHVSSYCGSGCTLHVSSYCLEVLFQGPGSGHHHHHH
(SEQ ID NO: 53)
MGSGKPTSTGQAGSGQQSWPISGSGHFYWMRGSGAEAAAREAAAREAAAREA
AAREAAAREAAAREAAAREAAAREAAAREAAAREAAAREAAAREAAAREAAAREAAAREAA
AREACAREAAAREAAAREAAAREAAAREAAAREAAAREAAAREAAAREAAAREAAAREAAA
REAAAREAAAREAAAREAAAREAAARAGSGHFYWMRGSGQQSWPISGSGKPTSTGQAENLYF
QGMGSGHHHHHH
(SEQ ID NO: 54)
MGSGCPTSTGQAGSGQQSWPISGSGHFYWMRGSGAEAAAREAAAREAAAREA
AAREAAAREAAAREAAAREAAAREAAAREAAAREAAAREAAAREAAAREAAAREAAAREAA
AREAKAREAAAREAAAREAAAREAAAREAAAREAAAREAAAREAAAREAAAREAAAREAAA
REAAAREAAAREAAAREAAAREAAARAGSGHFYWMRGSGQQSWPISGSGCPTSTGQAENLYF
QGMGSGHHHHHH
(SEQ ID NO: 55)
MGSGCPTSTGQAGSGQQSWPISGSGHFYWMRGSGAEAAAREAAAREAAAREA
AAREAAAREAAAREAAAREAAAREAAAREAAAREAAAREAAAREAAAREAAAREAAAREAA
AREAKAREAAAREAAAREAAAREAAAREAAAREAAAREAAAREAAAREAAAREAAAREAAA
REAAAREAAAREAAAREAAAREAAARAGSGHFYWMRGSGQQSWPISGSGCPTSTGQAENLYF
QGMGSDYKDDDDK
(SEQ ID NO: 56)
MGSGKPTSTGQAGSGQQSWPISGSGHFYWMRGSGAEAAAREAAAREAAAREA
AAREAAAREAAAREAAAREAAAREAAAREAAAREAAAREAAAREAAAREAAAREAAAREAA
AREACAREAAAREAAAREAAAREAAAREAAAREAAAREAAAREAAAREAAAREAAAREAAA
REAAAREAAAREAAAREAAAREAAARAGSGHFYWMRGSGQQSWPISGSGKPTSTGQAENLYF
QGMGSDYKDDDDK
(SEQ ID NO: 57)
HFYWMKGSGCTLHVSSYCGSGQQSWPISGSGTLHVSSYGSGAEAAAREAAARE
AAAREAAAREAAAREAAAREAAAREAAAREAAAREAAAREAAAREAAAREAAAREAAAREA
AAREAAAREAKAREAAAREAAAREAAAREAAAREAAAREAAAREAAAREAAAREAAAREAA
AREAAAREAAAREAAAREAAAREAAAREAAARAGSGTLHVSSYGSGQQSWPISGSGCTLHVSS
YCGSGKHFYWM
(SEQ ID NO: 58)
MDYKDDDDKGSGENLYFQGMGSGKHFYWMGSGQQSWPISGSGHFYWMGSGT
LHVSSYGSGHFYWMGSGAEALAREALAREALAREALAREALAREALAREALAREALAREALAR
EALAREALAREALAREALAREALAREALCREALAREALAREALAREALAREALAREALAREAL
AREALAREALAREALAREALAREALAREALAREALAREALAREALAREALARAGSGMHFYWG
SGTLHVSSYGSGMHFYWGSGQQSWPISGSGKMHFYWGSGHHHHHH
(SEQ ID NO: 59)
MDYKDDDDKGSGENLYFQGMGSGKHFYWMGSGQQSWPISGSGHFYWMGSGT
LHVSSYGSGHFYWMGSGAEALAREALAREALAREALAREALAREALAREALAREALAREALAR
EALAREALAREALAREALAREALAREALAREALAREALAREALAREALAREALAREALAREAL
AREALAREALAREALAREALAREALCREALAREALAREALAREALAREALAREALAREALARE
ALAREALAREALAREALAREALAREALAREALAREALAREALAREALAREALAREALAREALA
REALAREALAREALAREALAREALAREALARAGSGMHFYWGSGTLHVSSYGSGMHFYWGSGQ
QSWPISGSGKMHFYWGSGHHHHHH
(SEQ ID NO: 60)
QQSWPISGSGNFMSLPRLGHMHGSGTLHVSSYGSGAEAAAREAAAREAAAREA
AAREAAAREAAAREAAAREAAAREAAAREAAAREAAAREAAAREAAAREAAAREAAAREAA
AREACAREAAAREAAAREAAAREAAAREAAAREAAAREAAAREAAAREAAAREAAAREAAA
REAAAREAAAREAAAREAAAREAAARAGSGQQSWPISGSGNFMSLPRLGHMHGSGTLHVSSYG
SGENLYFQGMGSGHHHHHH
(SEQ ID NO: 61)
MGSGCCPGCCGSGQQSWPISGSGNFMSLPRLGHMHGSGAEAAAREAAAREAAA
REAAAREAAAREAAAREAAAREAAAREAAAREAAAREAAAREAAAREAAAREAAAREAAAR
EAAAREAAAREAAAREAAAREAAAREAKAREAAAREAAAREAAAREAAAREAAAREAAARE
AAAREAAAREAAAREAAAREAAAREAAAREAAAREAAAREAAAREAAAREAAAREAAAREA
AAREAAARAGSGNFMSLPRLGHMHGSGQQSWPISGSGCCPGCCGSGENLYFQGMGSGHHHHH
H

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A sensor device comprising:
a current carrying molecular structure comprising a metal contact on the surface of an electrode, wherein said metal contact is coupled to a bridge molecule,
wherein the bridge molecule comprises a peptide alpha helix having one or more metal binding motif selected from QQSWPIS (SEQ ID NO:2), CTLHVSSYC (SEQ ID NO:3), TLHVSSY (SEQ ID NO: 4), KGSGKGSGK (SEQ ID NO:5), CCPGCC (SEQ ID NO:6), CPTSTGQA (SEQ ID NO: 7), HFYWM (SEQ ID NO:8), HFYWMR (SEQ ID NO:9), PTSTGQA (SEQ ID NO:10), NFMSLPRLGHMH (SEQ ID NO:11) or any one of the preceding having one or two amino acid deletions or conservative amino acid substitutions; and
a probe molecule coupled to the bridge molecule;
wherein a circuit is formed that is capable of detecting and/or obtaining detailed information about the binding of the probe to a target ligand or binding partner.

2. A sensor device comprising:
a source electrode comprising a metal contact;
a drain electrode comprising a metal contact and spaced apart from the source electrode by a sensor gap, wherein the source and drain electrodes cooperate to form an electrode circuit;
a bridge molecule comprising a peptide alpha helix bridging across said sensor gap, coupling the source and drain electrodes, wherein the peptide alpha helix bridge molecule comprises one or more metal binding motif selected from QQSWPIS (SEQ ID NO:2), CTLHVSSYC (SEQ ID NO: 3), TLHVSSY (SEQ ID NO:4), KGSGKGSGK (SEQ ID NO:5), CCPGCC (SEQ ID NO:6), CPTSTGQA (SEQ ID NO:7), HFYWM (SEQ ID NO:8), HFYWMR (SEQ ID NO:9), PTSTGQA (SEQ ID NO:10), or NFMSLPRLGHMH (SEQ ID NO: 11); and
a probe coupled to the bridge molecule, wherein interaction of the probe with a target binding partner is detectable by monitoring at least one parameter of the electrode circuit detectable by the sensor.

3. A sensor device of claim 2, wherein the peptide alpha helix bridging further comprises one or more bioconjugation site for coupling the probe or a linker to the bridge molecule.

4. A sensor of claim 2, wherein the probe is a DNA polymerase.

5. (canceled)

6. A sensor device of claim 2, wherein the wherein the peptide alpha helix bridge molecule comprises SEQ ID NO:18 or SEQ ID NO:18 having up to three amino acid substitutions.

7. A sensor device of claim 6, comprising SEQ ID NO:18.

8. A sensor device of claim 2, wherein the peptide alpha helix bridge molecule comprises SEQ ID NO:19 or SEQ ID NO: 19 having up to three amino acid substitutions.

9. A sensor device of claim 8, comprising SEQ ID NO:19.

10. A sensor device of claim 2, wherein the wherein the peptide alpha helix bridge molecule comprises SEQ ID NO:20 or SEQ ID NO:20 having up to three amino acid substitutions.

11. A sensor device of claim 10, comprising SEQ ID NO:20.

12. A sensor device of claim 2, wherein the wherein the peptide alpha helix bridge molecule comprises SEQ ID NO:21 or SEQ ID NO:21 having up to three amino acid substitutions.

13. A sensor device of claim 12, comprising SEQ ID NO:21.

14. A sensor device of claim 2, wherein the wherein the peptide alpha helix bridge molecule comprises SEQ ID NO:22 or SEQ ID NO:22 having up to three amino acid substitutions.

15. A sensor device of claim 14, comprising SEQ ID NO:22.

16. A sensor device of claim 2, wherein the wherein the peptide alpha helix bridge molecule comprises SEQ ID NO:23 or SEQ ID NO:23 having up to three amino acid substitutions.

17. A sensor device of claim 16, comprising SEQ ID NO:23.

18. (canceled)

19. A method of detection of a target, the method comprising the steps of i) selecting a sensor according to any one of claims 1-4, or 6-17; ii) initiating at least one of a voltage or a current through the sensor; iii) exposing the sensor to a sample suspected of containing a target pf interest; iv) applied voltage on the sensor; and v) measuring an electrical change in the circuit.

20. A method of claim 19, used for determining the sequence of a target nucleic acid molecule in a sample suspected of containing the target.


SEQUENCE LISTING (63 sequences)

SEQ ID NO: 1 - protein, 8 aa, synthetic construct
ENLYFQGM

SEQ ID NO: 2 - protein, 7 aa, synthetic construct
QQSWPIS

SEQ ID NO: 3 - protein, 9 aa, synthetic construct
CTLHVSSYC

SEQ ID NO: 4 - protein, 7 aa, synthetic construct
TLHVSSY

SEQ ID NO: 5 - protein, 9 aa, synthetic construct
KGSGKGSGK

SEQ ID NO: 6 - protein, 6 aa, synthetic construct
CCPGCC

SEQ ID NO: 7 - protein, 8 aa, synthetic construct
CPTSTGQA

SEQ ID NO: 8 - protein, 5 aa, synthetic construct
HFYWM

SEQ ID NO: 9 - protein, 6 aa, synthetic construct
HFYWMR

SEQ ID NO: 10 - protein, 7 aa, synthetic construct
PTSTGQA

SEQ ID NO: 11 - protein, 12 aa, synthetic construct
NFMSLPRLGHMH

SEQ ID NO: 12 - protein, 5 aa, synthetic construct
EAAAR

SEQ ID NO: 13 - protein, 5 aa, synthetic construct
EAYAR

SEQ ID NO: 14 - protein, 5 aa, synthetic construct
AREAL

SEQ ID NO: 15 - protein, 4 aa, synthetic construct
YRAR

SEQ ID NO: 16 - protein, 5 aa, synthetic construct
YAREY

SEQ ID NO: 17 - skipped

SEQ ID NO: 18 - protein, 242 aa, synthetic construct
MDYKDDDDKGSGSGSQQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPIS

SEQ ID NO: 19 - protein, 351 aa, synthetic construct
MGSGQQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPISGSGENLYFQGMGSGHHHHHH

SEQ ID NO: 20 - protein, 298 aa, synthetic construct
MDYKDDDDKGSGENLYFQGMGSGKHFYWMGSGQQSWPISGSGHFYWMGSGTLHVSSYGSGFYWMGSGYEAYAREAYAREAYAREAAAREAAAREAAAREAYAREAYAREAYAREAAAREAAAREAAAREAYAREAYAREAYAREAAAREAAAREACAREAAAREAAAREAYAREAYAREAYAREAAAREAAAREAAAREAYAREAYAREAYAREAAAREAAAREAAAREAYAREAYAREAYARYGSGMHFYWGSGTLHVSSYGSGMHFYWGSGQQSWPISGSGKMHFYWGSGHHHHHH

SEQ ID NO: 21 - protein, 345 aa, synthetic construct
MDYKDDDDKGSGENLYFQGMGSGKHFYWMGSGQQSWPISGSGHFYWMGSGTLHVSSYGSGFYWMGSGAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTCLAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTALGSGMHFYWGSGTLHVSSYGSGMHFYWGSGQQSWPISGSGKMHFYWGSGHHHHHH

SEQ ID NO: 22 - protein, 281 aa, synthetic construct
ENLYFQGMGSGMGSGCTLHVSSYCGSGCTLHVSSYCGSGCTLHVSSYCGSGAAIPQFSAYRVRAYNSEAAAREAAAREAAARAIPQFSAYRVRAYNSEAAAREAAAREAAARAIPQFSAYRVRAYNSEAAAREAAAREAKAREAAAREAAARAIPQFSAYRVRAYNSEAAAREAAAREAAARAIPQFSAYRVRAYNSEAAAREAAAREAAARAIPQFSAYRVRAYNSAGSGCTLHVSSYCGSGCTLHVSSYCGSGCTLHVSSYCLEVLFQGPGSGHHHHHH

SEQ ID NO: 23 - protein, 351 aa, synthetic construct; identical to SEQ ID NO: 19

SEQ ID NO: 24 - protein, 217 aa, synthetic construct
MWKGSGKGSGKGSGGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGKGSGKGSGKGSGWENLYFQGMGSGHHHHHH

SEQ ID NO: 25 - protein, 242 aa, synthetic construct; identical to SEQ ID NO: 18

SEQ ID NO: 26 - protein, 247 aa, synthetic construct
QQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPISGSGENLYFQGMGSGHHHHHH

SEQ ID NO: 27 - protein, 263 aa, synthetic construct
MGSGCTLHVSSYCGSGCTLHVSSYCGSGCTLHVSSYCGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGCTLHVSSYCGSGCTLHVSSYCGSGCTLHVSSYCGSGENLYFQGMGSGHHHHHH

SEQ ID NO: 28 - protein, 291 aa, synthetic construct
MGSGQQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPISGSGENLYFQGMGSGHHHHHH

SEQ ID NO: 29 - protein, 363 aa, synthetic construct
MGSGCTLHVSSYCGSGCTLHVSSYCGSGCTLHVSSYCGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGCTLHVSSYCGSGCTLHVSSYCGSGCTLHVSSYCGSGENLYFQGMGSGHHHHHH

SEQ ID NO: 30 - protein, 291 aa, synthetic construct
MGSGTLHVSSYGSGTLHVSSYGSGTLHVSSYGSGAEAAAREAAAREAAAREAAAREAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAAREAAAREAAAREAAAREAAARAGSGTLHVSSYGSGTLHVSSYGSGTLHVSSYGSGENLYFQGMGSGHHHHHH

SEQ ID NO: 31 - protein, 351 aa, synthetic construct
MGSGTLHVSSYGSGTLHVSSYGSGTLHVSSYGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGTLHVSSYGSGTLHVSSYGSGTLHVSSYGSGENLYFQGMGSGHHHHHH

SEQ ID NO: 32 - protein, 256 aa, synthetic construct
MCGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGCGSGENLYFQGMGSGHHHHHH

SEQ ID NO: 33 - protein, 272 aa, synthetic construct
MKGSGKGSGKGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGKGSGKGSGKGSGENLYFQGMGSGHHHHHH

SEQ ID NO: 34 - protein, 212 aa, synthetic construct
MCTLHVSSYCGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGCTLHVSSYCGSGENLYFQGMGSGHHHHHH

SEQ ID NO: 35 - protein, 247 aa, synthetic construct
QQSWPISGSGQQSWPISGSGQQSWPISGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGQQSWPISGSGQQSWPISGSGQQSWPISGSGENLYFQGMGSGHHHHHH

SEQ ID NO: 36 - protein, 269 aa, synthetic construct
MGSGCCPGCCGSGQQSWPISGSGAEAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAARAGSGQQSWPISGSGCCPGCCGSGENLYFQGMGSGHHHHHH

SEQ ID NO: 37 - protein, 271 aa, synthetic construct
MGSGCCPGCCGSGTLHVSSYWGSGAEAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAARAGSGTLHVSSYWGSGCCPGCCGSGENLYFQGMGSGHHHHHH

SEQ ID NO: 38 - protein, 198 aa, synthetic construct
MWCGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGCGSGWENLYFQGMGSGHHHHHH

SEQ ID NO: 39 - protein, 258 aa, synthetic construct
MWCGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGCGSGWENLYFQGMGSGHHHHHH

SEQ ID NO: 40 - protein, 274 aa, synthetic construct
MWKGSGKGSGKGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGKGSGKGSGKGSGWENLYFQGMGSGHHHHHH

SEQ ID NO: 41 - protein, 383 aa, synthetic construct
WSHPQFEKENLYFQGMGSGCTLHVSSYCGSGQQSWPISGSGCTLHVSSYCGSGQQSWPISGSGAEAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAAREAAARAGSGQQSWPISGSGCTLHVSSYCGSGCTLHVSSYCGSGQQSWPISGSGHHHHHH

SEQ ID NO: 42 - protein, 253 aa, synthetic construct
WSHPQFEKENLYFQGMGSGCCPGCCGSGQQSWPISGSGQQSWPISGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGQQSWPISGSGQQSWPISGSGCCPGCCGSGHHHHHH

SEQ ID NO: 43 - protein, 253 aa, synthetic construct
WSHPQFEKENLYFQGMGSGCCPGCCGSGQQSWPISGSGQQSWPISGSGAEAAAREAAAREAAAREAAAREAAAREAAAREAAAREAAAREAAAREAAAREAAAREAAAREAAAREAAAREAAAREAAAREAYAREAAAREAAAREAAAREAAAREAAAREAAAREAAAREAAAREAAAREAAAREAAAREAAAREAAAREAAAREAAAREAAARAGSGQQSWPISGSGQQSWPISGSGCCPGCCGSGHHHHHH

SEQ ID NO: 44 - protein, 289 aa, synthetic construct
MDYKDDDDKGSGENLYFQGMGSGKHFYWMGSGQQSWPISGSGHFYWMGSGTLHVSSYGSGHFYWMGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGMHFYWGSGTLHVSSYGSGMHFYWGSGQQSWPISGSGKMHFYWGSGHHHHHH

SEQ ID NO: 45 - protein, 251 aa, synthetic construct
MEAAARMAAAFYAAWREAMARYEAFRGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAARAGSGEAAARMAAAFYAAWREAMARYEAFRDYKDDDDKGSGENLYFQGMGSGHHHHHH

SEQ ID NO: 46 - protein, 434 aa, synthetic construct
MDYKDDDDKGSGENLYFQGMGSGKQQSWPISGSGMQQSWPISGSGMQQSWPISGSGAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTCLAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTALAIPQFSAYRVKAYNSAASSDLRNLKTALGSGQQSWPISGSGMQQSWPISGSGKQQSWPISMGSGHHHHHH

SEQ ID NO: 47 - protein, 286 aa, synthetic construct
MDYKDDDDKGSGENLYFQGMGSGKFTLHVSSYFMGSGQQSWPISGSGFTLHVSSYFGSGMQQSWPISFEAYAREYREAYAREYAAREAAAREAAAREAYAREYREAYAREYAAAREAAAREAAAREAYAREYREAYAREYAAAREACAREAAAREAYAREYREAYAREYAAAREAAAREAAAREAYAREYREAYAREYAAAREAAAREAAAREAYAREYREAYAREYAFGSGQQSWPISMGSGFTLHVSSYFMGSGFTLHVSSYFMGSGKQQSWPISGSGHHHHHH

SEQ ID NO: 48 - protein, 287 aa, synthetic construct
MDYKDDDDKGSGENLYFQGMGSGKFTLHVSSYFMGSGQQSWPISGSGFTLHVSSYFGSGMQQSWPISFSYRARAYRARAYRARAAADAAADDAAADDAYRARAYRARAYRARAAADAAADDAAADDAYRARAYRARAYRARAAADAACDDAAADDAYRARAYRARAYRARAAADAAADDAAADDAYRARAYRARAYRARAAADAAADDAAADDAYRARAYRARAYRARSFGSGQQSWPISMGSGFTLHVSSYFMGSGFTLHVSSYFMGSGKQQSWPISGSGHHHHHH

SEQ ID NO: 49 - protein, 289 aa, synthetic construct
MDYKDDDDKGSGENLYFQGMGSGKHFYWMGSGQQSWPISGSGHFYWMGSGTLHVSSYGSGHFYWMGSGFSYRARAYRARAYRARAAAARRRREEEEAAAAYRARAYRARAYRARAAAARRRREEEEAAAAYRARAYRARAYRARAAAARRRCEEEAAAAYRARAYRARAYRARAAAARRRREEEEAAAAYRARAYRARAYRARAAAARRRREEEEAAAAYRARAYRARAYRARSFGSGMHFYWGSGTLHVSSYGSGMHFYWGSGQQSWPISGSGKMHFYWGSGHHHHHH

SEQ ID NO: 50 - protein, 267 aa, synthetic construct
MDYKDDDDKGSGENLYFQSGSGQQSWPISGSGQQSWPISGSGQQSWPISGSGAEAAAREAYAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAKAREAAAREAAAREAAAREAAAREAAAREAAARAGSGQQSWPISGSGQQSWPISGSGQQSWPISGSGLEVLFQGPKHHHHHH

SEQ ID NO: 51 - protein, 298 aa, synthetic construct; identical to SEQ ID NO: 20

SEQ ID NO: 52 - protein, 283 aa, synthetic construct
ENLYFQGMGSGMGSGCTLHVSSYCGSGCTLHVSSYCGSGCTLHVSSYCGSGAYEAYAREAYAREAYAREAAAREAAAREAAAREAYAREAYAREAYAREAAAREAAAREAAAREAYAREAYAREAYAREAAAREAAAREAKAREAAAREAAAREAYAREAYAREAYAREAAAREAAAREAAAREAYAREAYAREAYAREAAAREAAAREAAAREAYAREAYAREAYARYAGSGCTLHVSSYCGSGCTLHVSSYCGSGCTLHVSSYCLEVLFQGPGSGHHHHHH

SEQ ID NO: 53 - protein, 248 aa, synthetic construct
MGSGKPTSTGQAGSGQQSWPISGSGHFYWMRGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGHFYWMRGSGQQSWPISGSGKPTSTGQAENLYFQGMGSGHHHHHH

SEQ ID NO: 54 - protein, 248 aa, synthetic construct
MGSGCPTSTGQAGSGQQSWPISGSGHFYWMRGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGHFYWMRGSGQQSWPISGSGCPTSTGQAENLYFQGMGSGHHHHHH

SEQ ID NO: 55 - protein, 249 aa, synthetic construct
MGSGCPTSTGQAGSGQQSWPISGSGHFYWMRGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGHFYWMRGSGQQSWPISGSGCPTSTGQAENLYFQGMGSDYKDDDDK

SEQ ID NO: 56 - protein, 249 aa, synthetic construct
MGSGKPTSTGQAGSGQQSWPISGSGHFYWMRGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGHFYWMRGSGQQSWPISGSGKPTSTGQAENLYFQGMGSDYKDDDDK

SEQ ID NO: 57 - protein, 249 aa, synthetic construct
HFYWMKGSGCTLHVSSYCGSGQQSWPISGSGTLHVSSYGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGTLHVSSYGSGQQSWPISGSGCTLHVSSYCGSGKHFYWM

SEQ ID NO: 58 - protein, 284 aa, synthetic construct
MDYKDDDDKGSGENLYFQGMGSGKHFYWMGSGQQSWPISGSGHFYWMGSGTLHVSSYGSGHFYWMGSGAEALAREALAREALAREALAREALAREALAREALAREALAREALAREALAREALAREALAREALAREALAREALCREALAREALAREALAREALAREALAREALAREALAREALAREALAREALAREALAREALAREALAREALAREALAREALAREALARAGSGMHFYWGSGTLHVSSYGSGMHFYWGSGQQSWPISGSGKMHFYWGSGHHHHHH

SEQ ID NO: 59 - protein, 389 aa, synthetic construct
MDYKDDDDKGSGENLYFQGMGSGKHFYWMGSGQQSWPISGSGHFYWMGSGTLHVSSYGSGHFYWMGSGAEALAREALAREALAREALAREALAREALAREALAREALAREALAREALAREALAREALAREALAREALAREALAREALAREALAREALAREALAREALAREALAREALAREALAREALAREALAREALAREALCREALAREALAREALAREALAREALAREALAREALAREALAREALAREALAREALAREALAREALAREALAREALAREALAREALAREALAREALAREALAREALAREALAREALAREALAREALAREALARAGSGMHFYWGSGTLHVSSYGSGMHFYWGSGQQSWPISGSGKMHFYWGSGHHHHHH

SEQ ID NO: 60 - protein, 257 aa, synthetic construct
QQSWPISGSGNFMSLPRLGHMHGSGTLHVSSYGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGQQSWPISGSGNFMSLPRLGHMHGSGTLHVSSYGSGENLYFQGMGSGHHHHHH

SEQ ID NO: 61 - protein, 299 aa, synthetic construct
MGSGCCPGCCGSGQQSWPISGSGNFMSLPRLGHMHGSGAEAAAREAAAREAAAREAAAREAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAAREAAAREAAAREAAAREAAARAGSGNFMSLPRLGHMHGSGQQSWPISGSGCCPGCCGSGENLYFQGMGSGHHHHHH

SEQ ID NO: 62 - protein, 196 aa, synthetic construct
MCGSGAEAAAREAAAREAAAREAAAREAAAREAAAREAAAREAAAREAAAREAAAREAAAREAAAREAAAREAAAREAAAREAAAREAKAREAAAREAAAREAAAREAAAREAAAREAAAREAAAREAAAREAAAREAAAREAAAREAAAREAAAREAAAREAAAREAAARAGSGCGSGENLYFQGMGSGHHHHHH

SEQ ID NO: 63 - protein, 215 aa, synthetic construct
MKGSGKGSGKGSGGSGAEAAAREAAAREAAAREAAAREAAAREAAAREAAAREAAAREAAAREAAAREAAAREAAAREAAAREAAAREAAAREAAAREACAREAAAREAAAREAAAREAAAREAAAREAAAREAAAREAAAREAAAREAAAREAAAREAAAREAAAREAAAREAAAREAAARAGSGKGSGKGSGKGSGENLYFQGMGSGHHHHHH